(19) United States
(12) Patent Application Publication
(10) Pub. No.: US 2006/0247245 A1
(43) Pub. Date: Nov. 2, 2006

(54) Substituted imidazolopyrazine and triazolopyrazine derivatives: gabaa receptor ligands

(76) Inventor: Yuelian Xu, East Haven, CT (US)

Correspondence Address: CANTOR COLBURN, LLP, 55 GRIFFIN ROAD SOUTH, BLOOMFIELD, CT, 06002, US

(21) Appl. No.: 10/554,153

(22) PCT Filed: May 3, 2004
(86) PCT No.: PCT/US04/13778

§ 371 Date: Oct. 20, 2005

Related U.S. Application Data
(60) Provisional application No. 60/468,073, filed on May 5, 2003.

(51) Int. Cl. A61K 31/498; C07D 487/04

(52) U.S. Cl. 514/249; 544/349

(57) ABSTRACT

Compounds of Formula (1) are provided, as are methods for their preparation. The variables Z₁, Z₂, Z₃, R₄, R₅, R₆, R₇, R₈, and Ar in the above Formula are defined herein. Such compounds may be used to modulate ligand binding to GABA_{A} receptors in vivo or in vitro, and are particularly useful in the treatment of a variety of central nervous system (CNS) disorders in humans, domesticated companion animals, and livestock animals. Compounds provided herein may be administered alone or in combination with one or more other CNS agents to potentiate the effects of the other CNS agent(s). Pharmaceutical compositions and methods for treating such disorders are provided, as are methods for using such ligands for detecting GABA_{A} receptors (e.g., receptor localization studies).

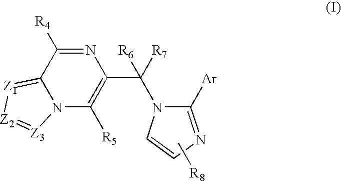

### FIELD OF THE INVENTION

The present invention relates generally to imidazolopyrazines and triazolopyrazines that have useful pharmacological properties. The present invention further relates to pharmaceutical compositions comprising such compounds and to the use of such compounds in the treatment of central nervous system (CNS) diseases.

### BACKGROUND OF THE INVENTION

The GABA_{A} receptor superfamily represents one of the classes of receptors through which the major inhibitory neurotransmitter γ-aminobutyric acid, or GABA, acts. Widely, although unequally, distributed throughout the mammalian brain, GABA mediates many of its actions through a complex of proteins called the GABA_{A} receptor, which causes alteration in chloride conductance and membrane polarization. A number of drugs, including the anxiolytic and sedating benzodiazepines, also bind to this receptor. The GABA_{A} receptor comprises a chloride channel that generally, but not invariably, opens' in response to GABA, allowing chloride to enter the cell. This, in turn, effects a slowing of neuronal activity through hyperpolarization of the cell membrane potential.

GABA_{A} receptors are composed of five protein subunits. A number of cDNAs for these GABA_{A} receptor subunits have been cloned and their primary structures determined. While these subunits share a basic motif of 4 membrane-spanning helices, there is sufficient sequence diversity to classify them into several groups. To date, at least 6α, 3β, 3γ, 1ε, 1δ and 2ρ subunits have been identified. Native GABA_{A} receptors are typically composed of 2 α subunits, 2 β subunits, and 1 γ subunit. Various lines of evidence (such as message distribution, genome localization and biochemical study results) suggest that the major naturally occurring receptor combinations are α₁β₂γ₂, α₂β₃γ₂, α₃β₃γ₂, and α₅β₃γ₂.

The GABA_{A} receptor binding sites for GABA (2 per receptor complex) are formed by amino acids from the α and β subunits. Amino acids from the α and γ subunits together form one benzodiazepine site per receptor, at which benzodiazepines exert their pharmacological activity. In addition, the GABA_{A} receptor contains sites of interaction for several other classes of drugs. These include a steroid binding site, a picrotoxin site, and a barbiturate site. The benzodiazepine site of the GABA_{A} receptor is a distinct site on the receptor complex that does not overlap with the site of interaction for other classes of drugs or GABA.

In a classic allosteric mechanism, the binding of a drug to the benzodiazepine site alters the affinity of the GABA receptor for GABA. Benzodiazepines and related drugs that enhance the ability of GABA to open GABA_{A} receptor channels are known as agonists or partial agonists, depending on the level of GABA enhancement. Other classes of drugs, such as β-carboline derivatives, that occupy the same site and negatively modulate the action of GABA are called inverse agonists. Those compounds that occupy the same site, and yet have little or no effect on GABA activity, can block the action of agonists or inverse agonists and are thus referred to as GABA_{A} receptor antagonists.

The important allosteric modulatory effects of drugs acting at the benzodiazepine site were recognized early, and the distribution of activities at different receptor subtypes has been an area of intense pharmacological discovery. Agonists that act at the benzodiazepine site are known to exhibit anxiolytic, sedative, anticonvulsant and hypnotic effects, while compounds that act as inverse agonists at this site elicit anxiogenic, cognition enhancing, and proconvulsant effects.

While benzodiazepines have enjoyed long pharmaceutical use as anxiolytics, these compounds can exhibit a number of unwanted side effects such as cognitive impairment, sedation, ataxia, potentiation of ethanol effects, and a tendency for tolerance and drug dependence. Accordingly, there is a need in the art for additional therapeutic agents that modulate GABA_{A} receptor activation and/or activity. The present invention fulfills this need, and provides further related advantages.

### SUMMARY OF THE INVENTION

The present invention provides compounds that modulate GABA_{A} receptor activation and/or GABA_{A} receptor-mediated signal transduction. Such GABA_{A} receptor modulators are preferably high affinity and/or high selectivity GABA_{A} receptor ligands and act as agonists, inverse agonists or antagonists of GABA_{A} receptors, such as human GABA_{A} receptors. As such, they are useful in the treatment of various CNS disorders.

Within certain aspects, GABA_{A} receptor modulators provided herein are substituted imidazolopyrazines and triazolopyrazines of Formula I: 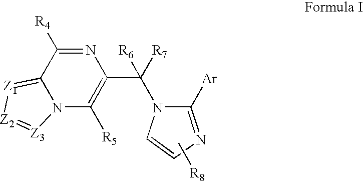
or pharmaceutically acceptable forms thereof, wherein:
Z₁ is nitrogen or CR₁; Z₂ is nitrogen or CR₂; Z₃ is nitrogen or CR₃; and at least one, but no more than two of Z₁, Z₂ and Z₃ are nitrogen;
Ar represents phenyl, naphthyl or 5- to 10-membered heteroaryl, each of which is optionally substituted, and preferably substituted with from 0 to 4 substituents independently chosen from halogen, hydroxy, nitro, cyano, amino, C₁-C₈alkyl, C₁-C₈alkenyl, C₁-C₈alkynyl, C₁-C₈alkoxy, C₃-C₇cycloalkyl, (C₃-C₇cycloalkyl)C₀-C₄alkyl, (C₃-C₇cycloalkyl)C₁-C₄alkoxy, C₁-C₈alkyl ether, C₁-C₈alkanone, C₁-C₈alkanoyl, 3- to 7-membered heterocycloalkyl, C₁-C₈haloalkyl, C₁-C₈haloalkoxy, oxo, C₁-C₈hydroxyalkyl, C₁-C₈aminoalkyl, and mono- and di-(C₁-C₈alkyl)amino(C₀-C₈alkyl);
R₁, R₂, R₃, and R₄ are each independently selected from:
   (a) hydrogen, halogen, nitro and cyano; and
   (b) groups of the formula: 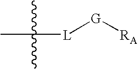
      wherein:
      L is a single covalent bond or C₁-C₈alkyl;
      G is a single covalent bond, N(R_{B}), O, C(═O), C(═O)O, C(═O)N(R_{B}), N(R_{B})C(═O), S(O)ₘ, CH₂C(═O), S(O)ₘN(R_{B}) or N(R_{B})S(O)ₘ; wherein m is 0, 1 or 2; and
      R_{A} and each R_{B} are independently selected from:
         (i) hydrogen; and
         (ii) C₁-C₈alkyl, C₂-C₈alkenyl, C₂-C₈alkynyl, (C₃-C₈cycloalkyl)C₀-C₄alkyl, (3- to 6-membered heterocycloalkyl)C₀-C₄alkyl, (aryl)C₀-C₂alkyl or (heteroaryl)C₀-C₂alkyl, each of which is optionally substituted, and preferably substituted with from 0 to 4 substituents independently selected from halogen, hydroxy, nitro, cyano, amino, C₁-C₄alkyl, C₁-C₄alkoxy, C₁-C₄alkanoyl, mono- and di(C₁-C₄alkyl)amino, C₁-C₄haloalkyl and C₁-C₄haloalkoxy;
R₅ is C₁-C₆alkyl, C₂-C₆alkenyl, C₁-C₄alkoxy, or mono- or di-(C₁-C₄alkyl)amino, each of which is substituted with from 0 to 5 substituents independently chosen from halogen, hydroxy, nitro, cyano, amino, C₁-C₄alkoxy, C₁-C₂haloalkyl, C₁-C₂haloalkoxy, mono- and di-C₁-C₄alkylamino, C₃-C₈cycloalkyl, phenylC₀-C₄alkyl and phenylC₁-C₄alkoxy;
R₆ and R₇ are independently hydrogen, halogen, methyl or ethyl; and
R₈ represents 0, 1 or 2 substituents independently chosen from halogen, hydroxy, nitro, cyano, amino, C₁-C₄alkyl, C₁-C₄alkoxy, mono- and di-C₁-C₄alkyl)amino, C₃-C₇Cycloalkyl, C₁-C₂haloalkyl and C₁-C₂haloalkoxy.

Within further aspects, the present invention provides pharmaceutical compositions comprising one or more compounds or forms thereof as described above in combination with a pharmaceutically acceptable carrier, diluent or excipient Packaged pharmaceutical preparations are also provided, comprising such a pharmaceutical composition in a container and instructions for using the composition to treat a patient suffering from a CNS disorder such as anxiety, depression, a sleep disorder, attention deficit disorder or Alzheimer's dementia.

The present invention further provides, within other aspects, methods for treating patients suffering from certain CNS disorders, such as anxiety, depression, a sleep disorder, attention deficit disorder, schizophrenia or Alzheimer's dementia, comprising administering to a patient in need of such treatment a GABA_{A} receptor modulatory amount of a compound or pharmaceutically acceptable form thereof as described above. Methods for improving short term memory in a patient are also provided, comprising administering to a patient in need of such treatment a GABA_{A} receptor modulatory amount of a compound or pharmaceutically acceptable form thereof as described above. Treatment of humans, domesticated companion animals (pets) or livestock animals suffering from certain CNS disorders with an effective amount of a compound of the invention is encompassed by the present invention.

In a separate aspect, the invention provides methods of potentiating the actions of other CNS active compounds. These methods comprise administering a GABA_{A} receptor modulatory amount of a compound or pharmaceutically acceptable form thereof of Formula I in conjunction with the administration of another CNS active compound.

The present invention further relates to the use of compounds of Formula I as probes for the localization of GABA_{A} receptors in sample (e.g., a tissue section). In certain embodiments, GABA_{A} receptors are detected using autoradiography. Additionally, the present invention provides methods for determining the presence or absence of GABA_{A} receptor in a sample, comprising the steps of: (a) contacting a sample with a compound as described above under conditions that permit binding of the compound to GABA_{A} receptor; (b) removing compound that does not bind to the GABA_{A} receptor and (c) detecting a level of compound bound to GABA_{A} receptor.

In yet another aspect, the present invention provides methods for preparing the compounds disclosed herein, including the intermediates.

These and other aspects of the present invention will become apparent upon reference to the following detailed description.

### DETAILED DESCRIPTION OF THE INVENTION

As noted above, the present invention provides substituted imidazolopyrazines and triazolopyrazines of Formula I, including imidazo[1,2-a]pyrazines, imidazo[1,5-a]pyrazines, [1,2,4]triazolo[4,3-a]pyrazines and [1,2,4]triazolo[1,5-a]pyrazines. Certain preferred compounds bind to GABA_{A} receptor, preferably with high selectivity. Certain preferred compounds further provide beneficial modulation of brain function. Without wishing to be bound to any particular theory of operation, it is believed that that interaction of such compounds with the benzodiazepine site of GABA_{A} receptor results in the pharmacological effects of these compounds. Such compounds may be used in vitro or in vivo to determine the location of GABA_{A} receptors or to modulate GABA_{A} receptor activity in a variety of contexts.

### Chemical Description and Terminology

Compounds provided herein are generally described using standard nomenclature. For compounds having asymmetric centers, it should be understood that (unless otherwise specified) all of the optical isomers and mixtures thereof are encompassed. All chiral (enantiomeric and diastereomeric), and racemic forms, as well as all geometric isomeric forms of a structure are intended, unless the specific stereochemistry or isomeric form is specifically indicated. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. Recited compounds are further intended to encompass compounds in which one or more atoms are replaced with an isotope (i.e., an atom having the same atomic number but a different mass number). By way of general example, and without limitation, isotopes of hydrogen include tritium and deuterium and isotopes of carbon include ¹¹C, ¹³C, and ¹⁴C.

Certain compounds are described herein using a general formula that includes variables. Unless otherwise specified, each variable within such a formula is defined independently of other variables, and any variable that occurs more than one time within a formula is defined independently at each occurrence. Thus, for example, if a group is described as being substituted with 0-2 R, then the group may be unsubstituted or substituted with up to two R* groups and R* at each occurrence is selected independently from the definition of R*. In addition, it will be apparent that combinations of substituents and/or variables are permissible only if such combinations result in stable compounds.

The phrase “substituted imidazolopyrazines and triazolopyrazines” as used herein, refers to compounds of Formula I, as well as pharmaceutically acceptable forms thereof.

“Pharmaceutically acceptable forms” of the compounds recited herein include pharmaceutically acceptable salts, esters, hydrates, clathrates and prodrugs of such compounds, as well as all crystalline forms. As used herein, a pharmaceutically acceptable salt is an acid or base salt that is generally considered in the art to be suitable for use in contact with the tissues of human beings or animals without excessive toxicity, irritation, allergic response, or other problem or complication, commensurate with a reasonable benefit/risk ratio. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable salt can be synthesized from a parent compound that contains a basic or acidic moiety by any conventional chemical method, such as by reacting a free acid or base form of the compound with a stoichiometric amount of an appropriate base or acid in water, an organic solvent, or a mixture of the two; generally, nonaqueous media such as ether, ethyl acetate, ethanol, isopropanol or acetonitrile are preferred.

A “prodrug” is a compound that may not fully satisfy the structural requirements of Formula L but is modified in vivo, following administration to a patient, to produce a compound of Formula I. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxyl, amino, or sulfhydryl group, respectively. Examples of prodrugs include, but are not limited to, acetate, formate and benzoate derivatives of alcohol and amine functional groups within the compounds provided herein. Prodrugs of the compounds of Formula I may be prepared, for example, by modifying functional groups present in the compounds in such a way that the modifications are cleaved in vivo to a compound of Formula I.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity). When a substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone.

The phrase “optionally substituted” indicates that a group may either be unsubstituted or substituted at one or more of any of the available positions, typically 1, 2, 3, 4 or 5 positions, by one or more suitable substituents such as those disclosed herein. Optional substitution is also indicated by the phrase “substituted with from 0 to X substituents,” in which X is the maximum number of substituents. Suitable substituents include, for example, halogen, cyano, amino, hydroxy, nitro, azido, carboxamido, —COOH, SO₂NH₂, alkyl (e.g. C₁-C₈alkyl), alkenyl (e.g., C₂-C₈alkenyl), alkynyl (e.g., C₂-C₈alkynyl), alkoxy (e.g. C₁-C₈alkoxy), alkyl ether (e.g., C₂-C₈alkyl ether), alkylthio (e.g., C₁-C₈alkylthio), haloalkyl (e.g., C₁-C₈haloalkyl), hydroxyalkyl (e.g., C₁-C₈hydroxyalkyl), aminoalkyl (e.g., C₁-C₈aminoalkyl), haloalkoxy (e.g., C₁-C₈haloalkoxy), alkanoyl (e.g., C₁-C₈alkanoyl), alkanone (e.g., C₁-C₈alkanone), alkanoyloxy (e.g., C₁-C₈alkanoyloxy), alkoxycarbonyl (e.g. C₁-C₈alkoxycarbonyl), mono- and di-(C₁-C₈alkyl)amino, mono- and di-(C₁-C₈alkyl)aminoC₁-C₈alkyl, mono- and di-(C₁-C₈alkyl)carboxamido, mono- and di-(C₁-C₈alkyl)sulfonamido, alkylsulfinyl (e.g., C₁-C₈alkylsulfinyl), alkylsulfonyl (e.g., C₁-C₈alkylsulfonyl), aryl (e.g., phenyl), arylalkyl (e.g., (C₆-C₁₈aryl)C₁-C₈alkyl, such as benzyl and phenethyl), aryloxy (e.g., C₆-C₁₈aryloxy such as phenoxy), arylalkoxy (e.g., (C₆-C₁₈aryl)C₁-C₈alkoxy) and/or 3- to 8-membered heterocyclic groups such as coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino or pyrrolidinyl. Certain groups within the formulas provided herein are optionally substituted with from 1 to 3, 1 to 4 or 1 to 5 independently selected substituents.

A dash (“−”) that is not between two letters or symbols is used to indicate a point of attachment for a substituent. For example, —CONH₂ is attached through the carbon atom.

As used herein, “alkyl” is intended to include both branched and straight-chain saturated aliphatic hydrocarbon groups, and where specified, having the specified number of carbon atoms. Thus, the term C₁-C₆alkyl, as used herein, indicates an alkyl group having from 1 to 6 carbon atoms. “C₀,” as used herein, refers to a single covalent bond; for example, “C₀-C₄alkyl” refers to a single covalent bond or a C₁-C₄alkyl group. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl, and 3-methylpentyl. In certain embodiments, preferred alkyl groups are methyl, ethyl, propyl, butyl, and 3-pentyl. “Aminoalkyl” is an alkyl group as defined herein substituted with one or more —NH₂ substituents. “Hydroxyalkyl” is a hydroxy group as defined herein substituted with one or more —OH substituents.

“Alkenyl” refers to a straight or branched hydrocarbon chain comprising one or more unsaturated carbon-carbon bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more triple carbon-carbon bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include for example groups such as ethynyl and propynyl.

A “cycloalkyl” is a saturated cyclic group in which all ring members are carbon, such as cyclopropyl, cyclobutyl, cyclopentyl, and cyclohexyl. Such groups typically contain from 3 to about 8 ring carbon atoms; in certain embodiments, such groups have from 3 to 7 ring carbon atoms. Examples of cycloalkyl groups include cyclopropyl, cyclobutyl, cyclopentyl, or cyclohexyl and bridged or caged saturated ring groups such as norbornane or adamantane and the like. If substituted, any ring carbon atom may be bonded to any indicated substituent, such as halogen, cyano, C₁-C₈alkyl, C₁-C₈alkoxy, or C₂-C₈alkanoyl.

In the term “(cycloalkyl)alkyl”, “cycloalkyl” and “alkyl” are as defined above, and the point of attachment is on the alkyl group. This term encompasses, but is not limited to, cyclopropylmethyl, cyclohexylmethyl and cyclohexylethyl. The term “(C₃-C₇cycloalkyl)C₀-C₄alkyl” refers to a 3- to 7-membered cycloalkyl group linked via a single covalent bond or a C₁-C₄alkyl group. Similarly, the term “(C₃-C₇cycloalkyl)C₁-C₄alkoxy” refers to a 3- to 7-membered cycloalkyl group linked via a C₁-C₄alkoxy group.

By “alkoxy,” as used herein, is meant an alkyl, alkenyl or alkynyl group as described above attached via an oxygen bridge. Alkoxy groups include C₁-C₆alkoxy and C₁-C₄alkoxy groups, which have from 1 to 6 or 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are specific alkoxy groups. Similarly “alkylthio” refers to an alkyl, alkenyl or alkynyl group as described above attached via a sulfur bridge.

As used herein, the term “alkylsulfinyl” refers to groups of the formula —SO)-alkyl, in which the sulfur atom is the point of attachment. Alkylsulfinyl groups include C₁-C₆alkylsulfinyl and C₁-C₄alkylsulfinyl groups, which have from 1 to 6 or 1 to 4 carbon atoms, respectively.

“Alkylsulfonyl” refers to groups of the formula —(SO₂)-alkyl, in which the sulfur atom is the point of attachment. Alkylsulfonyl groups include C₁-C₆alkylsulfonyl and C₁-C₄alkylsulfonyl groups, which have from 1 to 6 or 1 to 4 carbon atoms, respectively. Methylsulfonyl is one representative alkylsulfonyl group.

“Alkylsulfonamido” refers to groups of the formula —(SO₂)—NR₂, in which the sulfur atom is the point of attachment and each R is independently hydrogen or alkyl. The term “mono- or di-(C₁-C₆alkyl)sulfonamido” refers to such groups in which one R is C₁-C₆alkyl and the other R is hydrogen or an independently chosen C₁-C₆alkyl.

The term “alkanoyl” refers to an alkyl group as defined above attached through a carbonyl bridge. Alkanoyl groups include C₂-C₈alkanoyl, C₂-C₆alkanoyl and C₂-C₄alkanoyl groups, which have from 2 to 8, 2 to 6 or 2 to 4 carbon atoms, respectively. “C₁alkanoyl” refers to —C═O)—H, which (along with C₂-C₈alkanoyl) is encompassed by the term “C₁-C₈alkanoyl.” Ethanoyl is C₂alkanoyl.

The term “oxo,” as used herein, refers to a keto (C═O) group. An oxo group that is a substituent of a nonaromatic ring results in a conversion of —CH₂— to —C(═O). It will be apparent that the introduction of an oxo substituent on an aromatic ring destroys the aromaticity.

An “alkanone” is an alkyl group as defined above with the indicated number of carbon atoms substituted at least one position with an oxo group. “C₃-C₈alkanone,” “C₃-C₆alkanone” and “C₃-C₄alkanone” refer to an alkanone having from 3 to 8, 6 or 4 carbon atoms, respectively. By way of example, a C₃ alkanone group has the structure —CH₂—(C═O)—CH₃.

Similarly, “alkyl ether” refers to a linear or branched ether substituent linked via a carbon-carbon bond. Alkyl ether groups include C₂-C₈alkyl ether, C₂-C₆alkyl ether and C₂-C₄alkyl ether groups, which have 2 to 8, 6 or 4 carbon atoms, respectively. By way of example, a C₂ alkyl ether group has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₂-C₈, C₂-C₆ and C₂-C₄alkoxycarbonyl groups, which have from 2 to 8, 6 or 4 carbon atoms, respectively. “C₁alkoxycarbonyl” refers to —C(═O)—OH, which is encompassed by the term “C₁-C₈alkoxycarbonyl.” Such groups may also be referred to as alkylcarboxylate groups. For example, methyl carboxylate refers to —C(═O)—O—CH₃ and ethyl carboxylate refers to —C(═O)—O—CH₂CH₃.

“Alkylamino” refers to a secondary or tertiary amine having the general structure —NH-alkyl or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups.

“Alkylaminoalkyl” refers to an alkylamino group linked via an alkyl group (i.e., a group having the general structure -alkyl-NH-alkyl or -alkyl-N(alkyl)(alkyl)) in which each alkyl is selected independently. Such groups include, for example, mono- and di-(C₁-C₈alkyl)aminoC₁-C₈alkyl, mono- and di-(C₁-C₆alkyl)aminoC₁-C₆alkyl and mono- and di-(C₁-C₄alkyl)aminoC₁-C₄alkyl, in which each alkyl may be the same or different. “Mono- or di-(C₁-C₈alkyl)aminoC₀-C₈alkyl” refers to a mono- or di-(C₁-C₈alkyl)amino group hiked via a single covalent bond or a C₁-C₈alkyl group. Examples of such group include methylaminomethyl and diethylaminomethyl, as well as the following:

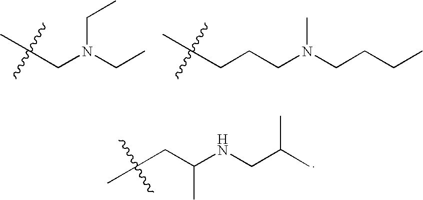

The term “carboxamido” refers to an amide group (i.e., —(C═O)NH₂).

The term “halogen” refers to fluorine, chlorine, bromine or iodine.

A “haloalkyl” is a branched or straight-chain alkyl group, substituted with 1 or more halogen atoms (e.g., “haloC₁-C₈alkyl” groups have from 1 to 8 carbon atoms; “haloC₁-C₆alkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; and mono-, di-, tri-, tetra- or penta-chloroethyl. Typical haloalkyl groups are trifluoromethyl and difluoromethyl. Within certain compounds provided herein, not more than 5 or 3 haloalkyl groups are present. The term “haloalkoxy” refers to a haloalkyl group as defined above attached via an oxygen bridge. “HaloC₁-C₈alkoxy” groups have 1 to 8 carbon atoms.

The term “carbocycle” or “carbocyclic group” is used herein to indicate saturated, partially unsaturated or aromatic groups having 1 ring or 2 fused, pendant or spiro rings, with 3 to 8 atoms in each ring, wherein all ring atoms are carbon. A carbocyclic group may be bound through any carbon atom that results in a stable structure, and may be substituted on any carbon atom if the resulting compound is stable. Carbocyclic groups include cycloalkyl, cycloalkenyl, and aryl groups. Bicyclic carbocyclic groups may have 1 cycloalkyl ring and 1 partially unsaturated or aromatic ring (e.g., a tetrahydronapthyl group).

As used herein, the term “aryl” indicates aromatic groups containing only carbon in the aromatic ring(s). Such aromatic groups may be further substituted with carbon or non-carbon atoms or groups. Typical aryl groups contain 1 to 3 separate, fused, Spiro or pendant rings and from 6 to about 18 ring atoms, without heteroatoms as ring members. Representative aryl groups include phenyl, naphthyl (including 1-naphthyl and 2-naphthyl) and biphenyl. The term (aryl)C₀-C₂alkyl” refers to an aryl group (preferably a C₆-C₁₀aryl group) that is linked via a single covalent bond, methyl or ethyl. The term “phenylC₀-C₄alkyl” refers to a phenyl group linked via a single covalent bond or a C₁-C₄alkyl group. Similarly, the term “phenylC₁-C₄alkoxy” refers to a phenyl group linked via a C₁-C₄alkoxy group.

A “heteroatom” is an atom other than carbon, such as oxygen, sulfur or nitrogen.

The term “heterocycle” or “heterocyclic group” is used to indicate saturated, partially unsaturated, or aromatic groups having 1 or 2 rings, with 3 to 8 atoms in each ring, and in at least one ring from 1 to 4 heteroatoms independently selected from N, O and S. The heterocyclic ring may be attached at any heteroatom or carbon atom that results in a stable structure, and may be substituted on carbon and/or nitrogen atom(s) if the resulting compound is stable. Any nitrogen and/or sulfur heteroatoms may optionally be oxidized, and any nitrogen may optionally be quaternized. Bicyclic heterocyclic groups may, but need not, contain 1 saturated ring and 1 partially unsaturated or aromatic ring (e.g., a tetrahydroquinolinyl group).

Certain heterocycles are “heteroaryl” (i.e., groups that comprise at least one aromatic ring having from 1 to 4 heteroatoms), such as 5- to 10-membered heteroaryl groups (e.g., 5-to 7-membered monocyclic groups or 7- to 10-membered bicyclic groups). When the total number of S and 0 atoms in the heteroaryl group exceeds 1, then these heteroatoms are not adjacent to one another, preferably the total number of S and 0 atoms in the heteroaryl is not more than 1, 2 or 3, more preferably 1 or 2 and most preferably not more than 1. Examples of heteroaryl groups include pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl and 5,6,7,8-tetrahydroisoquinoline. A “5- or 6-membered heteroaryl” is a monocyclic heteroaryl having 5 or 6 ring members. The term (heteroaryl)C₀-C₂alkyl” refers to a heteroaryl group (preferably a 5- to 10-membered heteroaryl group) that is linked via a single covalent bond, methyl or ethyl.

Other heterocycles are referred to herein as “heterocycloalkyl” (i.e., saturated heterocycles). Heterocycloalkyl groups have from 3 to about 8 ring atoms, and more typically from 3 to 7 or from 5 to 7 ring atoms. Examples of heterocycloalkyl groups include morpholinyl, piperazinyl and pyrrolidinyl. The term “(3- to 6-membered heterocycloalkyl)C₀-C₄alkyl” refers to a heterocycloalkyl groups having from 3 to 6 ring atoms, linked via a single covalent bond or a C₁-C₄alkyl group.

The terms “GABA_{A} receptor” and “benzodiazepine receptor” refer to a protein complex that detectably binds GABA and mediates a dose dependent alteration in chloride conductance and membrane polarization. Receptors comprising naturally-occurring mammalian (especially human or rat) GABA_{A} receptor subunits are generally preferred, although subunits may be modified provided that any modifications do not substantially inhibit the receptor's ability to bind GABA (i.e., at least 50% of the binding affinity of the receptor for GABA is retained). The binding affinity of a candidate GABA_{A} receptor for GABA may be evaluated using a standard ligand binding assay as provided herein. It will be apparent that there are a variety of GABA_{A} receptor subtypes that fall within the scope of the term “GABA_{A} receptor.” These subtypes include, but are not limited to, α₂β₃γ₂, α₃β₃γ₂, α₅β₃γ₂, and α₁β₂γ₂ receptor subtypes. GABA_{A} receptors may be obtained from a variety of sources, such as from preparations of rat cortex or from cells expressing cloned human GABA_{A} receptors. Particular subtypes may be readily prepared using standard techniques (e.g., by introducing mRNA encoded the desired subunits into a host cell, as described herein).

An “agonist” of a GABA_{A} receptor is a compound that enhances the activity of GABA at the GABA_{A} receptor. Agonists may, but need not, also enhance the binding of GABA to GABA_{A} receptor. The ability of a compound to act as a GABA_{A} agonist may be determined using an electrophysiological assay, such as the assay provided in Example 11.

An “inverse agonist” of a GABA_{A} receptor is a compound that reduces the activity of GABA at the GABA_{A} receptor. Inverse agonists, but need not, may also inhibit binding of GABA to the GABA_{A} receptor. The reduction of GABA-induced GABA_{A} receptor activity may be determined from an electrophysiological assay such as the assay of Example 11.

An “antagonist” of a GABA_{A} receptor, as used herein, is a compound that occupies the benzodiazepine site of the GABA_{A} receptor, but has no detectable effect on GABA activity at the GABA_{A} receptor. Such compounds can inhibit the action of agonists or inverse agonists. GABA_{A} receptor antagonist activity may be determined using a combination of a suitable GABA_{A} receptor binding assay, such as the assay provided in Example 10, and a suitable functional assay, such as the electrophysiological assay provided in Example 11, herein.

A “GABA_{A} receptor modulator” is any compound that acts as a GABA_{A} receptor agonist, inverse agonist or antagonist. In certain embodiments, such a modulator may exhibit an affinity constant of less than 1 micromolar in a standard GABA_{A} receptor radioligand binding assay, or an EC₅₀ of less than 1 micromolar in an electrophysiological assay as provided in Example 11. In other embodiments a GABA_{A} receptor modulator may exhibit an affinity constant or EC₅₀ of less than 500 nM, 200 nM, 100 nM, 50 nM, 25 nM, 10 nM or 5 nM.

A “GABA_{A} receptor modulatory amount” is an amount of GABA_{A} receptor modulator that results in an effective concentration of modulator at a target GABA_{A} receptor. An effective concentration is a concentration that is sufficient to result in a statistically significant (i.e., p≦0.05, which is determined using a conventional parametric statistical analysis method such as a student's T-test) inhibition of total specific binding of ³H-Flumazenil within the assay described in Example 10.

A GABA_{A} receptor modulator is said to have “high affinity” if the Kᵢ at a GABA_{A} receptor is less than 1 micromolar, preferably less than 100 nanomolar or less than 10 nanomolar. A representative assay for determining Kᵢ at the GABA_{A} receptor is provided in Example 10, herein. It will be apparent that the Kᵢ may depend upon the receptor subtype used in the assay. In other words, a high affinity compound may be “subtype-specific” (i.e., the Kᵢ is at least 10-fold greater for one subtype than for another subtype). Such compounds are said to have high affinity for GABA_{A} receptor if the Kᵢ for at least one GABA_{A} receptor subtype meets the above criteria.

A GABA_{A} receptor modulator is said to have “high selectivity” if it binds to a GABA_{A} receptor with a Kᵢ that is at least 10-fold lower, preferably at least 100-fold lower, than the Kᵢ for binding to other membrane-bound receptors. In particular, the compound should have a Kᵢ that is at least 10-fold greater at the following receptors than at a GABA_{A} receptor: serotonin, dopamine, galanin, VR₁, C5a, MCH, NPY, CRF, bradykinin and tackykinin. Assays to determine Kᵢ at other receptors may be performed using standard binding assay protocols, such as using a commercially available membrane receptor binding assay (e.g., the binding assays available from MDS PHARMA SERVICES, Toronto, Canada and CEREP, Redmond, Wash.).

A “patient” is any individual treated with a compound provided herein. Patients include humans, as well as other animals such as companion animals and livestock. Patients may be afflicted with a CNS disorder, or may be free of such a condition (i.e., treatment may be prophylactic).

A “CNS disorder” is a disease or condition of the central nervous system that is responsive to GABA_{A} receptor modulation in the patient Such disorders include anxiety disorders (e.g., panic disorder, obsessive compulsive disorder, agoraphobia, social phobia, specific phobia, dysthymia, adjustment disorders, separation anxiety, cyclothymia, and generalized anxiety disorder), stress disorders (e.g., post-traumatic stress disorder, anticipatory anxiety acute stress disorder and acute stress disorder), depressive disorders (e.g., depression, atypical depression, bipolar disorder and depressed phase of bipolar disorder), sleep disorders (e.g., primary insomnia, circadian rhythm sleep disorder, dyssomnia NOS, parasomnias including nightmare disorder, sleep terror disorder, sleep disorders secondary to depression, anxiety and/or other mental disorders and substance-induced sleep disorder), cognitive disorders (e.g., cognition impairment, mild cognitive impairment (MCI), age-related cognitive decline (ARCD), schizophrenia, traumatic brain injury, Down's Syndrome, neurodegenerative diseases such as Alzheimer's disease and Parkinson's disease, and stroke), AIDS-associated dementia, dementia associated with depression, anxiety or psychosis, attention deficit disorders (e.g., attention deficit disorder and attention deficit and hyperactivity disorder), convulsive disorders (e.g., epilepsy), benzodiazepine overdose and drug and alcohol addiction.

A “CNS agent” is any drug used to treat or prevent a CNS disorder. CNS agents include, for example: serotonin receptor (e.g., 5-HT_{1A}) agonists and antagonists and selective serotonin reuptake inhibitors (SSRIs); neurokinin receptor antagonists; corticotropin releasing factor receptor (CRF₁) antagonists; melatonin receptor agonists; nicotinic agonists; muscarinic agents; acetylcholinesterase inhibitors and dopamine receptor agonists.

Substituted Imidazolopyrazine and Triazolopyrazine Derivatives

As noted above, the present invention provides compounds or Formula I, with the variables as described above, as well as pharmaceutically acceptable forms of such compounds.

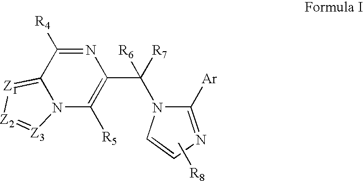

In certain embodiments, Ar represents phenyl, pyridyl, thiazolyl, thienyl, triazolopyridyl, pyridizinyl or pyrimidinyl, each of which is substituted with from 0 to 4 substituents as described above (e.g., 0, 1, 2 or 3 substituents independently chosen from, halogen, hydroxy, amino, cyano C₁-C₄alkyl, C₁-C₄alkoxy, mono- and di-(C₁-C₄alkyl)amino, C₂-C₄alkanoyl, (C₃-C₇cycloalkyl)C₀-C₂alkyl, C₁-C₂haloalkyl and C₁-C₂haloalkoxy). Representative Ar moieties include, for example, phenyl, pyridyl (e.g., pyridine-2-yl), thiazolyl (e.g., 1,3-thiazol-2-yl), thienyl (e.g., 2-thienyl), pyridizinyl (e.g., pyridizin-3-yl) and triazolopyridyl (e.g., [1,2,4]triazolo[4,3-a]pyridin-5-yl), each of which is substituted with from 0 to 3 substituents independently chosen from chloro, fluoro, hydroxy, cyano, amino, C₁-C₄alkyl, C₁-C₄alkoxy, C₁-C₂alkylamino, C₁-C₂haloalkyl, and C₁-C₂haloalkoxy. In certain embodiments, Ar represents pyridin-2-yl, 3-fluoropyridin-2-yl, 6-fluoro-pyridin-2-yl, 2,6-difluorophenyl, 2,5-difluorophenyl, 3-fluorophenyl or 3-methyl-[1,2,4]triazolo[4,3-a]pyridin-5-yl.

R₈ represents 0, 1, or 2 substituents independently chosen from halogen, hydroxy, nitro, cyano, amino, C₁-C₄alkyl, C₁-C₄alkoxy, mono- and di-(C₁-C₄alkyl)amino, C₃-C₇cycloalkyl, C₁-C₂haloalkyl, and C₁-C₂haloalkyoxy; in certain embodiments, R₈ represents 0 or 1 substituent chosen from halogen, C₁-C₂alkyl and C₁-C₂alkoxy.

As noted above, R₁, R₂, R₃ and R₄ are each independently selected from: (a)hydrogen, halogen, nitro and cyano; and (b) groups of the formula: 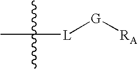
wherein: L represents a single covalent bond (i.e., L is absent) or C₁-C₈alkyl; G is a single covalent bond (i.e., L is directly linked to R_{A} via a single bond), 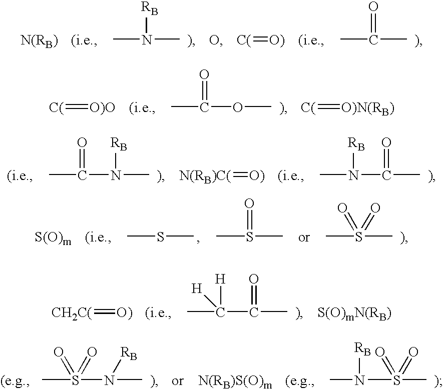
wherein m is 0, 1 or 2; and R_{A} and each R_{B} are as described above.

R₁, R₂, R₃ and R₄, in certain embodiments, are each independently selected from: (a) hydrogen, halogen and cyano; and (b) groups of the formula: 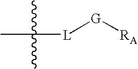
wherein: (i) L is a single covalent bond, methylene or ethylene; (ii) G is a single covalent bond, NH, N(R_{B}), O, C(═O)O or C(═O); and (iii) R_{A} and R_{B} (if present) are independently selected from (1) hydrogen and (2) C₁-C₆alkyl, C₂-C₆alkenyl, C₃-C₇cycloalkyl, 4- to 7-membered heterocycloalkyl, phenyl, thienyl, pyridyl, pyrimidinyl, thiazolyl, imidazolyl, pyrazolyl, pyridazinyl and pyrazinyl, each of which is substituted with from 0 to 4 substituents independently selected from hydroxy, halogen, cyano, amino, C₁-C₂alkyl and C₁-C₂alkoxy. Representative R₁, R₂, R₃ and R₄ groups include hydrogen, hydroxy, halogen, cyano, C₁-C₆alkyl, C₁-C₆alkoxy, C₃-C₇cycloalkyl, C₁-C₂alkoxyC₁-C₄alkyl, C₁-C₄hydroxyalkyl, C₁-C₂haloalkyl, C₁-C₂haloalkoxy, mono- and di-(C₁-C₄alkyl)amino, phenyl and pyridyl. In certain embodiments, R₁ and R₄ are independently hydrogen, methyl or ethyl. R₃, in certain embodiments, is chosen from hydrogen, C₁-C₄alkyl, C₁-C₄alkoxy, C₃-C₇cycloalkyl, C₁-C₂alkoxyC₁-C₂alkyl, C₁-C₂hydroxyalkyl, trifluoromethyl, phenyl, and pyridyl.

Within certain embodiments, Ar and R₈ are as described above, Z₁ is nitrogen and Z₂ is CR₂. Representative R₂, R₃ and R₄ groups within such compounds include, for example, hydrogen, halogen, C₁-C₄alkyl and C₁-C₄alkoxy. In other embodiments, Ar and R₈ are as described above, Z₁ is CR₁ and Z₂ is nitrogen. Representative R₁, R₃ and R₄ within such compounds include, for example, hydrogen, halogen, C₁-C₄alkyl and C₁-C₄alkoxy. Within further embodiments, Ar and R₈ are as described above and Z₁ and Z₂ are nitrogen. Representative R₃ and R₄ groups within such compounds include, for example, hydrogen, halogen, C₁-C₄alkyl and C₁-C₄alkoxy.

In certain compounds provided herein, R₅ is C₁-C₆ alkyl, C₂-C₆ alkenyl, C₁-C₄ alkoxy, or mono- or di-C₁-C₄alkylamino (preferably C₁-C₆ alkyl or C₂-C₆ alkenyl), each of which is substituted with from 0 to 2 substituents independently chosen from halogen, hydroxy, C₁-C₂alkoxy, C₃-C₈cycloalkyl, phenylC₀-C₂alkyl and phenylC₁-C₂alkoxy. R₅ groups include, for example, ethyl, propyl, butyl, ethoxy and methoxymethyl.

R₆ and R₇ are generally hydrogen, halogen or lower alkyl; in certain embodiments, both are hydrogen.

Within certain compounds, R₁, R₂, R₃ and R₄ are independently selected from:

(a) hydrogen, halogen and cyano; and

(b) groups of the formula: 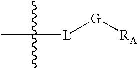
wherein:
(i) L is a single covalent bond;
(ii) G is a single covalent bond, NH, N(R_{B}), O, C(═O)O or C(═O); and
(iii) R_{A} and R_{B} are independently selected from (1) hydrogen and (2) C₁-C₆alkyl, C₂-C₆alkenyl, (C₃-C₇cycloalkyl)C₀-C₂alkyl, phenyl, thienyl, pyridyl, pyrimidinyl, thiazolyl and pyrazinyl, each of which is substituted with from 0 to 4 substituents independently selected from hydroxy, halogen, cyano, amino, C₁-C₂alkyl and C₁-C₂alkoxy.

In certain such compounds, R₁, R₂, R₃ and R₄ are independently selected from hydrogen, hydroxy, halogen, cyano, C₁-C₆alkyl, C₁-C₆alkoxy, C₃-C₇cycloalkyl, C₁-C₂alkoxyC₁-C₄alkyl, C₁-C₄hydroxyalkyl, C₁-C₂haloalkyl, C₁-C₂haloalkoxy, C₁-C₄carboxylate, mono- and di-(C₁-C₄alkyl)amino, phenylC₀-C₁alkyl, pyridylC₀-C₁alkyl and (4- to 7-membered heterocycloalkyl)C₀-C₁alkyl. Within one category of such compounds, R₁ and R₄ are independently chosen from hydrogen, methyl and ethyl.

As noted above, Z₁ is nitrogen or CR₁; Z₂ is nitrogen or CR₂ and Z₃ is nitrogen or CR₃. In certain compounds, Z₁ is nitrogen, Z₂ is CR₂ and Z₃ is CR₃. Such compounds include those in which R₂, R₃ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenyl and pyridyl). In other compounds, Z₁ is CR₁, Z₂ is nitrogen and Z₃ is CR₃. Such compounds include those in which R₁, R₃ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenyl and pyridyl). Within still further compounds, Z₁ and Z₂ are nitrogen and Z₃ is CR₃. Such compounds include those in which R₃ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenyl and pyridyl. In other compounds, wherein Z₁ and Z₃ are nitrogen and Z₂ is CR₂. Such compounds include those in which R₂ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenyl and pyridyl.

Compounds provided herein detectably alter (modulate) ligand binding to GABA_{A} receptor, as determined using a standard in vitro receptor binding assay. References herein to a “GABA_{A} receptor ligand binding assay” are intended to refer to the standard in vitro receptor binding assay provided in Example 10. Briefly, a competition assay may be performed in which a GABA_{A} receptor preparation is incubated with labeled (e.g., ³H) ligand, such as Flumazenil, and unlabeled test compound. Incubation with a compound that detectably modulates ligand binding to GABA_{A} receptor will result in a decrease or increase in the amount of label bound to the GABA_{A} receptor preparation, relative to the amount of label bound in the absence of the compound. Preferably, such a compound will exhibit a K; at GABA_{A} receptor of less than 1 micromolar, more preferably less than 500 nM, 100 nM, 20 nM or 10 nM. The GABA_{A} receptor used to determine in vitro binding may be obtained from a variety of sources, for example from preparations of rat cortex or from cells expressing cloned human GABA_{A} receptors.

In certain embodiments, preferred compounds have favorable pharmacological properties, including oral bioavailability (such that a sub-lethal or preferably a pharmaceutically acceptable oral dose, preferably less than 2 grams, more preferably less than or equal to one gram or 200 mg, can provide a detectable in vivo effect), low toxicity (a preferred compound is nontoxic when a GABA_{A} receptor-modulatory amount is administered to a subject), minimal side effects (a preferred compound produces side effects comparable to placebo when a GABA_{A} receptor-modulatory amount of the compound is administered to a subject), low serum protein binding, and a suitable in vitro and in vivo half-life (a preferred compound exhibits an in vitro half-life that is equal to an in vivo half-life allowing for Q.I.D. dosing, preferably T.I.D. dosing, more preferably B.I.D. dosing and most preferably once-a-day dosing). Distribution in the body to sites of complement activity is also desirable (e.g., compounds used to treat CNS disorders will preferably penetrate the blood brain barrier, while low brain levels of compounds used to treat periphereal disorders are typically preferred).

Routine assays that are well known in the art may be used to assess these properties and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays, such as those described by Oravcova, et al. (1996) Journal of Chromatography B 677:1-27. Compound half-life is inversely proportional to the frequency of dosage of a compound required to achieve an effective amount In vitro half-lives of compounds may be predicted from assays of microsomal half-life as described by Kuhnz and Gieschen (1998) Drug Metabolism and Disposition 26:1120-27.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement and (4) does not cause substantial release of liver enzymes.

As used herein, a compound that “does not substantially inhibit cellular ATT production” is a compound that, when tested as described in Example 12, does not decrease cellular ATP levels by more than 50%. Preferably, cells treated as described in Example 12 exhibit ATP levels that are at least 80% of the ATP levels detected in untreated cells. The concentration of modulator used in such assays is generally at least 10-fold, 100-fold or 1000-fold greater than the EC₅₀ or IC₅₀ for the modulator in the assay of Example 11.

A compound that “does not significantly prolong heart QT intervals” is a compound that does not result in a statistically significant prolongation of heart QT intervals (as determined by electrocardiography) in guinea pigs, minipigs or dogs upon administration of twice the minimum dose yielding a therapeutically effective in vivo concentration. In certain preferred embodiments, a dose of 0.01, 0.05. 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound “does not cause substantial liver enlargement” if daily treatment of laboratory rodents (e.g., mice or rats) for 5-10 days with twice the minimum dose that yields a therapeutically effective in vivo concentration results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound “does not promote substantial release of liver enzymes” if administration of twice the minimum dose yielding a therapeutically effective in vivo concentration does not elevate serum levels of ALT, LDH or AST in laboratory rodents by more than 3-fold (preferably no more than 2-fold) over matched mock-treated controls. In more highly preferred embodiments, such doses do not elevate such serum levels by more than 75% or 50% over matched controls. Alternately, a compound “does not promote substantial release of liver enzymes” if, in an in vitro hepatocyte assay, concentrations (in culture media or other such solutions that are contacted and incubated with hepatocytes in vitro) equivalent to two-fold the minimum in vivo therapeutic concentration of the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the minimum in vivo therapeutic concentration of the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the minimum therapeutically effective in vivo concentration.

Certain preferred compounds are not clastogenic or mutagenic (e.g. as determined using standard assays such as the Chinese hamster ovary cell vitro micronucleus assay, the mouse lymphoma assay, the human lymphocyte chromosomal aberration assay, the rodent bone marrow micronucleus assay, the Ames test or the like) at a concentration equal to the minimum therapeutically effective in vivo concentration. In other embodiments, certain preferred compounds do not induce sister chromatid exchange (e.g., in Chinese hamster ovary cells) at such concentrations.

For detection purposes, as discussed in more detail below, compounds provided herein may be isotopically-labeled or radiolabeled. Such compounds are identical to those described above, but for the fact that one or more atoms are replaced by an atom having an atomic mass or mass number different from the atomic mass or mass number usually found in nature. Examples of isotopes that can be incorporated into compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, such as increased in vivo half-life or reduced dosage requirements and, hence, may be preferred in some circumstances.

As noted above, different stereoisomeric forms, such as racemates and optically active forms, are encompassed by the present invention. In certain embodiments, it may be desirable to obtain single enantiomers (i.e., optically active forms). Standard methods for preparing single enantiomers include asymmetric synthesis and resolution of the racemates. Resolution of the racemates can be accomplished by conventional methods such as crystallization in the presence of a resolving agent, or chromatography using, for example, a chiral HPLC column.

Pharmaceutical Compositions

The present invention also provides pharmaceutical compositions comprising at least one GABA_{A} receptor modulator provided herein, in combination with at least one physiologically acceptable carrier or excipient. Such compounds may be used for treating patients in which GABA_{A} receptor modulation is desirable (e.g. patients undergoing painful procedures who would benefit from the induction of amnesia, or those suffering from anxiety, depression, sleep disorders or cognitive impairment). Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs or cats). If desired, other active ingredients may also be included, such as additional CNS-active agents.

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active materials in admixture with one or more excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also contain one or more preservatives, for example ethyl, or n-propyl p-hydroxybenzoate, one or more coloring agents, one or more flavoring agents and/or one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. One or more sweetening agents and/or flavoring agents may be added to provide palatable oral preparations. Such suspension may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be in the form of oil-in-water emulsions. The oily phase may be a vegetable oil (e.g., olive oil or arachis oil) or a mineral oil (e.g., liquid paraffin) or mixtures thereof. Suitable emulsifying agents may be naturally-occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). The emulsions may also contain sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectible aqueous or oleaginous suspension. The compound, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Pharmaceutical compositions may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

For administration to non-human animals, the composition may also be added to animal feed or drinking water. It may be convenient to formulate animal feed and drinking water compositions so that the animal takes in an appropriate quantity of the composition along with its diet. It may also be convenient to present the composition as a premix for addition to feed or drinking water.

Pharmaceutical compositions may be formulated as sustained release formulations (i.e., a formulation such as a capsule that effects a slow release of compound following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of active compound release. The amount of compound contained within a sustained release formulation depends upon the site of implantation, the rate and expected duration of release and the nature of the condition to be treated or prevented.

Compounds provided herein are generally present within a pharmaceutical composition in a therapeutically effective amount A therapeutically effective amount is an amount that results in a discernible patient benefit, such as diminution of symptoms of a CNS disorder. A preferred concentration is one sufficient to inhibit the binding of GABA_{A} receptor ligand to GABA_{A} receptor in vitro. Compositions providing dosage levels ranging from about 0.1 mg to about 140 mg per kilogram of body weight per day are preferred (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. It will be understood, however, that the optimal dose for any particular patient will depend upon a variety of factors, including the activity of the specific compound employed; the age, body weight, general health, sex and diet of the patient, the time and route of administration; the rate of excretion; any simultaneous treatment, such as a drug combination; and the type and severity of the particular disease undergoing treatment. Optimal dosages may be established using routine testing and procedures that are well known in the art.

Pharmaceutical compositions may be packaged for treating a CNS disorder such as anxiety, depression, a sleep disorder, attention deficit disorder or Alzheimer's dementia. Packaged pharmaceutical preparations include a container holding a therapeutically effective amount of at least one compound as described herein and instructions (e.g., labeling) indicating that the contained composition is to be used for treating the CNS disorder.

Methods of Use

Within certain aspects, the present invention provides methods for inhibiting the development of a CNS disorder. In other words, therapeutic methods provided herein may be used to treat an existing disorder, or may be used to prevent, decrease the severity of, or delay the onset of such a disorder in a patient who is free of detectable CNS disorder. CNS disorders are discussed in more detail below, and may be diagnosed and monitored using criteria that have been established in the art Alternatively, or in addition, compounds provided herein may be administered to a patient to improve short-term memory. Patients include humans, domesticated companion animals (pets, such as dogs) and livestock animals, with dosages and treatment regimes as described above.

Frequency of dosage may vary, depending on the compound used and the particular disease to be treated or prevented. In general, for treatment of most disorders, a dosage regimen of 4 times daily or less is preferred. For the treatment of sleep disorders a single dose that rapidly reaches effective concentrations is desirable. Patients may generally be monitored for therapeutic effectiveness using assays suitable for the condition being treated or prevented, which will be familiar to those of ordinary skill in the art.

Within preferred embodiments, compounds provided herein are used to treat patients in need of such treatment. In general, such patients are treated with a GABA_{A} receptor modulatory amount of a compound of Formula I (or a pharmaceutically acceptable form thereof), preferably the amount is sufficient to alter one or more symptoms of a CNS disorder. Compounds that act as agonists at α₂β₃γ₂ and α₃β₃γ₂ receptor subtypes are particularly useful in treating anxiety disorders such as panic disorder, obsessive compulsive disorder and generalized anxiety disorder; stress disorders including post-traumatic stress and acute stress disorders. Compounds that act as agonists at α₂β₃γ₂ and α₃β₃γ₂ receptor subtypes are also useful in treating depressive or bipolar disorders, schizophrenia and sleep disorders, and may be used in the treatment of age-related cognitive decline and Alzheimer's disease. Compounds that act as inverse agonists at the α₅β₃γ₂ receptor subtype or α₁β₂γ₂ and α₅β₃γ₂ receptor subtypes are particularly useful in treating cognitive disorders including those resulting from Down's Syndrome, neurodegenerative diseases such as Alzheimer's disease, Parkinson's disease and stroke related dementia. Compounds that act as inverse agonists at the α₅β₃γ₂ receptor subtype are particularly useful in treating cognitive disorders through the enhancement of memory, particularly short-term memory, in memory-impaired patients; while those that act as agonists at the α₅β₃γ₂ receptor subtype are particularly useful for the induction of amnesia. Compounds that act as agonists at the α₁β₂γ₂ receptor subtype are useful in treating convulsive disorders such as epilepsy. Compounds that act as antagonists at the benzodiazepine site are useful in reversing the effect of benzodiazepine overdose and in treating drug and alcohol addiction.

CNS disorders that can be treated using compounds and compositions provided herein include:
Depression, e.g., depression, atypical depression, bipolar disorder, depressed phase of bipolar disorder.
Anxiety, e.g., general anxiety disorder (GAD), agoraphobia, panic disorder +/−agoraphobia, social phobia, specific phobia, Post traumatic stress disorder, obsessive compulsive disorder (OCD), dysthymia, adjustment disorders with disturbance of mood and anxiety, separation anxiety disorder, anticipatory anxiety acute stress disorder, adjustment disorders, cyclothymia.
Sleep disorders, e.g., sleep disorders including primary insomnia, circadian rhythm sleep disorder, dyssomnia NOS, parasomnias, including nightmare disorder, sleep terror disorder, sleep disorders secondary to depression and/or anxiety or other mental disorders, substance induced sleep disorder.
Cognition Impairment e.g., cognition impairment, Alzheimer's disease, Parkinson's disease, mild cognitive impairment (MCI), age-related cognitive decline (ARCD), stroke, traumatic brain injury, AIDS associated dementia, and dementia associated with depression, anxiety and psychosis (including schizophrenia and hallucinatory disorders).
Attention Deficit Disorder. e.g., attention deficit disorder (ADD) and attention deficit and hyperactivity disorder (ADHD).
Speech disorders. e.g., motor tic, clonic stuttering, dysfluency, speech blockage, dysardria, Tourette's Syndrome and logospasm.

Compounds and compositions provided herein can also be used to improve short-term memory (working memory) in a patient. A therapeutically effective amount of a compound for improving short-term memory loss is an amount sufficient to result in a statistically significant improvement in any standard test of short-term memory function, including forward digit span and serial rote learning. For example, such a test may be designed to evaluate the ability of a patient to recall words or letters. Alternatively, a more complete neurophysical evaluation may be used to assess short-term memory function. Patients treated in order to improve short-term memory may, but need not, have been diagnosed with memory impairment or considered predisposed to development of such impairment

In a separate aspect, the present invention provides methods for potentiating the action (or therapeutic effect) of other CNS agent(s). Such methods comprise administering a GABA_{A} receptor modulatory amount of a compound provided herein in combination with another CNS agent. Such CNS agents include, but are not limited to the following: for anxiety, serotonin receptor (e.g., 5-HT_{1A}) agonists and antagonists; for anxiety and depression, neurokinin receptor antagonists or corticotropin releasing factor receptor (CRF₁) antagonists; for sleep disorders, melatonin receptor agonists; and for neurodegenerative disorders, such as Alzheimer's dementia, nicotinic agonists, muscarinic agents, acetylcholinesterase inhibitors and dopamine receptor agonists. Within certain embodiments, the present invention provides a method of potentiating the antidepressant activity of selective serotonin reuptake inhibitors (SSRIs) by administering an effective amount of a GABA agonist compound provided herein in combination with an SSRI. An effective amount of compound is an amount sufficient to result in a detectable change in patient symptoms, when compared to a patient treated with the other CNS agent alone. Combination administration can be carried out using well known techniques (e.g., as described by Da-Rocha, et al. (1997) J. Psychopharmacology 11(3):211-218; Smith, et al. (1998) Am. J. Psychiatry 155(10):133945; and Le, et al. (1996) Alcohol and Alcoholism 31(suppl.):127-132. See also PCT International Publication Nos. WO 99/47142; WO 99/47171; WO 99/47131 and WO 99/37303.

The present invention also pertains to methods of inhibiting the binding of benzodiazepine compounds (i.e., compounds that comprise the benzodiazepine ring structure), such as RO15-1788 or GABA, to GABA_{A} receptor. Such methods involve contacting a GABA_{A} receptor modulatory amount of a compound provided herein with cells expressing GABA_{A} receptor. This method includes, but is not limited to, inhibiting the binding of benzodiazepine compounds to GABA_{A} receptors in vivo (e.g., in a patient given an amount of a GABA_{A} receptor modulator provided herein that would be sufficient to inhibit the binding of benzodiazepine compounds or GABA to GABA_{A} receptor in vitro). In one embodiment, such methods are useful in treating benzodiazepine drug overdose. The amount of GABA_{A} receptor modulator that is sufficient to inhibit the binding of a benzodiazepine compound to GABA_{A} receptor may be readily determined via a GABA_{A} receptor binding assay as described in Example 10.

Within separate aspects, the present invention provides a variety of in vitro uses for the GABA_{A} receptor modulators provided herein. For example, such compounds may be used as probes for the detection and localization of GABA_{A} receptors, in samples such as tissue sections, as positive controls in assays for receptor activity, as standards and reagents for determining the ability of a candidate agent to bind to GABA_{A} receptor, or as radiotracers for positron emission tomography (PET) imaging or for single photon emission computerized tomography (SPECT). Such assays can be used to characterize GABA_{A} receptors in living subjects. Such compounds are also useful as standards and reagents in determining the ability of a potential pharmaceutical to bind to GABA_{A} receptor.

Within methods for determining the presence or absence of GABA_{A} receptor in a sample, a sample may be incubated with a GABA_{A} receptor modulator as provided herein under conditions that permit binding of the GABA_{A} receptor modulator to GABA_{A} receptor. The amount of GABA_{A} receptor modulator bound to GABA_{A} receptor in the sample is then detected. For example, a GABA_{A} receptor modulator may be labeled using any of a variety of well known techniques (e.g., radiolabeled with a radionuclide such as tritium, as described herein), and incubated with the sample (which may be, for example, a preparation of cultured cells, a tissue preparation or a fraction thereof). A suitable incubation time may generally be determined by assaying the level of binding that occurs over a period of time. Following incubation, unbound compound is removed, and bound compound detected using any method suitable for the label employed (e.g., autoradiography or scintillation counting for radiolabeled compounds; spectroscopic methods may be used to detect luminescent groups and fluorescent groups). As a control, a matched sample may be simultaneously contacted with radiolabeled compound and a greater amount of unlabeled compound. Unbound labeled and unlabeled compound is then removed in the same fashion, and bound label is detected. A greater amount of detectable label in the test sample than in the control indicates the presence of GABA_{A} receptor in the sample. Detection assays, including receptor autoradiography (receptor mapping) of GABA_{A} receptors in cultured cells or tissue samples may be performed as described by Kuhar in sections 8.1.1 to 8.1.9 of Current Protocols in Pharmacology (1998) John Wiley & Sons, New York.

For example, GABA_{A} receptor modulators provided herein may be used for detecting GABA_{A} receptors in cell or tissue samples. This may be done by preparing a plurality of matched cell or tissue samples, at least one of which is prepared as an experimental sample and at least one of which is prepared as a control sample. The experimental sample is prepared by contacting (under conditions that permit binding of RO15-1788 to GABA_{A} receptors within cell and tissue samples) at least one of the matched cell or tissue samples that has not previously been contacted with any GABA_{A} receptor modulator provided herein with an experimental solution comprising a detectably-labeled preparation of the selected GABA_{A} receptor modulator at the first measured molar concentration. The control sample is prepared in the same manner as the experimental sample and also contains an unlabelled preparation of the same compound at a greater molar concentration.

The experimental and control samples are then washed to remove unbound detectably-labeled compound. The amount of remaining bound detectably-labeled compound is then measured and the amount of detectably-labeled compound in the experimental and control samples is compared. The detection of a greater amount of detectable label in the washed experimental sample(s) than in control sample(s) demonstrates the presence of GABA_{A} receptor in the experimental sample.

The detectably-labeled GABA_{A} receptor modulator used in this procedure may be labeled with a radioactive label or a directly or indirectly luminescent label. When tissue sections are used in this procedure and the label is a radiolabel, the bound, labeled compound may be detected autoradiographically.

Compounds provided herein may also be used within a variety of well known cell culture and cell separation methods. For example, compounds may be linked to the interior surface of a tissue culture plate or other cell culture support, for use in immobilizing GABA_{A} receptor-expressing cells for screens, assays and growth in culture. Such linkage may be performed by any suitable technique, such as the methods described above, as well as other standard techniques. Compounds may also be used to facilitate cell identification and sorting in vitro, permitting the selection of cells expressing a GABA_{A} receptor. Preferably, the compound(s) for use in such methods are labeled as described herein. Within one preferred embodiment, a compound linked to a fluorescent marker, such as fluorescein, is contacted with the cells, which are then analyzed by fluorescence activated cell sorting (FACS).

Within other aspects, methods are provided for modulating binding of ligand to a GABA_{A} receptor in vitro or in vivo, comprising contacting a GABA_{A} receptor with a sufficient amount of a GABA_{A} receptor modulator provided herein, under conditions suitable for binding of ligand to the receptor. The GABA_{A} receptor may be present in solution, in a cultured or isolated cell preparation or within a patient. Preferably, the GABA_{A} receptor is a present in the brain of a mammal. In general, the amount of compound contacted with the receptor should be sufficient to modulate ligand binding to GABA_{A} receptor in vitro within, for example, a binding assay as described in Example 10.

Also provided herein are methods for altering the signal-transducing activity of cellular GABA_{A} receptor (particularly the chloride ion conductance), by contacting GABA_{A} receptor, either in vitro or in vivo, with a sufficient amount of a compound as described above, under conditions suitable for binding of Flumazenil to the receptor. The GABA_{A} receptor may be present in solution, in a cultured or isolated cell or cell membrane preparation or within a patient, and the amount of compound may be an amount that would be sufficient to alter the signal-transducing activity of GABA_{A} receptor in vitro. In certain embodiments, the amount of compound contacted with the receptor should be sufficient to modulate Flumazenil binding to GABA_{A} receptor in vitro within, for example, a binding assay as described in Example 10. An effect on signal-transducing activity may be assessed as an alteration in the electrophysiology of the cells, using standard techniques. The amount of a compound that would be sufficient to alter the signal-transducing activity of GABA_{A} receptors may be determined via a GABA_{A} receptor signal transduction assay, such as the assay described in Example 11. The cells expressing the GABA receptors in vivo may be, but are not limited to, neuronal cells or brain cells. Such cells may be contacted with compounds of the invention through contact with a body fluid containing the compound, for example through contact with cerebrospinal fluid. Alteration of the signal-transducing activity of GABA_{A} receptors in cells in vitro may be determined from a detectable change in the electrophysiology of cells expressing GABA_{A} receptors, when such cells are contacted with a compound of the invention in the presence of GABA.

Intracellular recording or patch-clamp recording may be used to quantitate changes in electrophysiology of cells. A reproducible change in behavior of an animal given a compound of the invention may also be taken to indicate that a change in the electrophysiology of the animal's cells expressing GABA_{A} receptors has occurred.

Preparation of Compounds

Compounds provided herein may generally be prepared using standard synthetic methods. Starting materials are generally readily available from commercial sources, such as Sigma-Aldrich Corp. (St. Louis, Mo.), or may be prepared as described herein. Representative procedures suitable for the preparation of compounds of Formula I are outlined in the following Schemes, which are not to be construed as limiting the invention in scope or spirit to the specific reagents and conditions shown in them. Those having skill in the art will recognize that the reagents and conditions may be varied and additional steps employed to produce compounds encompassed by the present invention. In some cases, protection of reactive functionalities may be necessary to achieve the desired transformations. In general, such need for protecting groups, as well as the conditions necessary to attach and remove such groups, will be apparent to those skilled in the art of organic synthesis. Unless otherwise stated in the schemes below, the variables are as defined in Formula I.

Abbreviations used the following Schemes and the accompanying Examples are as follows:

Ac acetate

Ac₂O acetic anhydride

BINAP 2,2′-bis(diphenylphosphino)-1,1′-binaphthyl

CDCl₃ deuterated chloroform

δ chemical shift

DCM dichloromethane

DMF N,N-dimethylformamide

EtOAc ethyl acetate

EtOH ethanol

HPLC high pressure liquid chromatography

¹H NMR proton nuclear magnetic resonance

Hz hertz

LC/MS liquid chromatography/mass spectrometry

mCPBA m-chloroperoxybenzoic acid

MeOH methanol

MS mass spectrometry

M+1 mass+1

mCPBA m-chloroperoxybenzoic acid

Ph Phenyl

Pd(PPh₃)₄ Tetrakis(triphenylphosphine)palladium(0)

Pd(Ph₃P)₂Cl₂ dichlorobis(triphenylphosphine)palladium (II)

PTLC preparative thin layer chromatography

THF tetrahydrofuran

TLC thin layer chromatography
Reaction Schemes

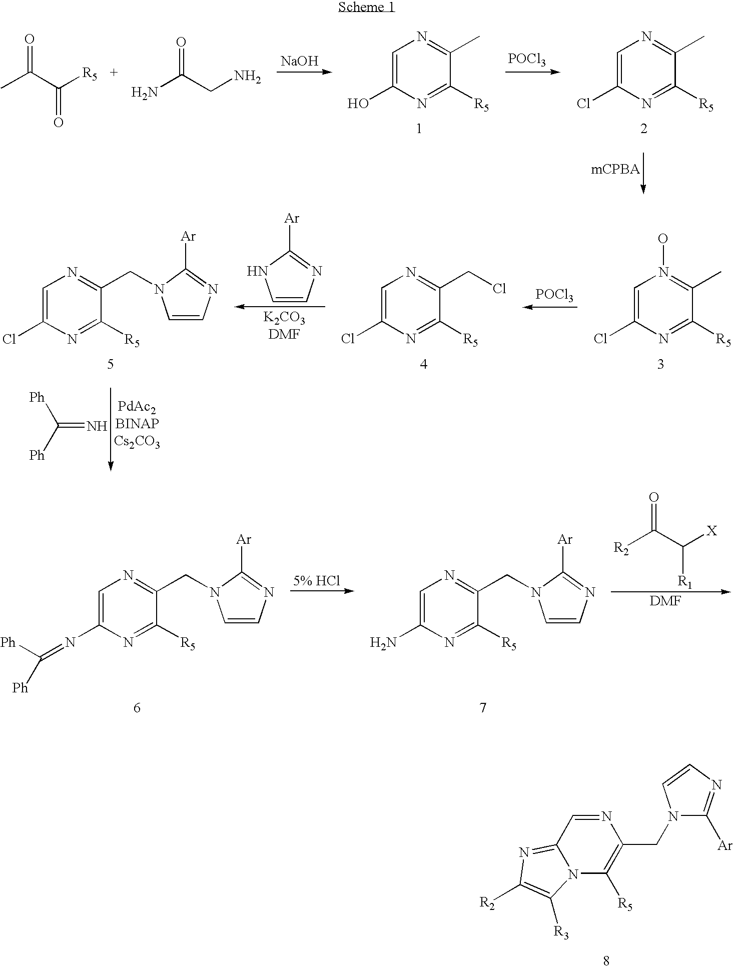

Scheme 1 illustrates the synthesis of imidazole fused pyrazines 8. Hydroxypyrazine 1 is prepared essentially according to J. Am. Chem. Soc. 74:1580 (1952), and is converted to chloropyrazine 2 upon treatment with POCl₃. mCPBA treatment of 2 selectively oxidizes the nitrogen meta to the chlorine, providing 3. 3 reacts with POCl₃ to produce chloromethyl derivative 4, which couples with an aryl substituted imidazole to give 5. Amination of 5 under Pd coupling conditions followed by acid cleavage provides 7, which condenses with an α-haloaldehyde or ketone to afford the product 8.

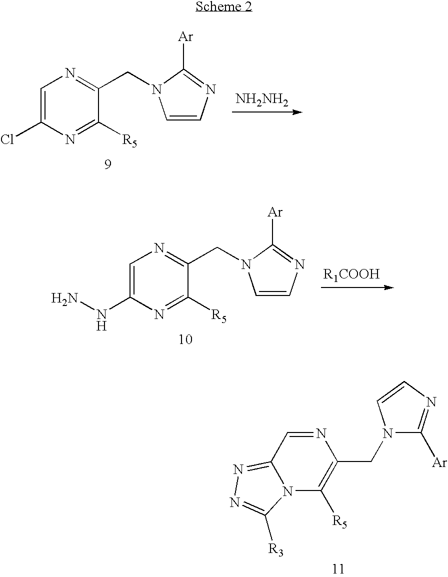

Scheme 2 illustrates the synthesis of triazole fused pyrazines 11. Treatment of hydrazine with chloropyrazine 9 affords 10, which upon refluxing with a carboxylic acid provides 11.

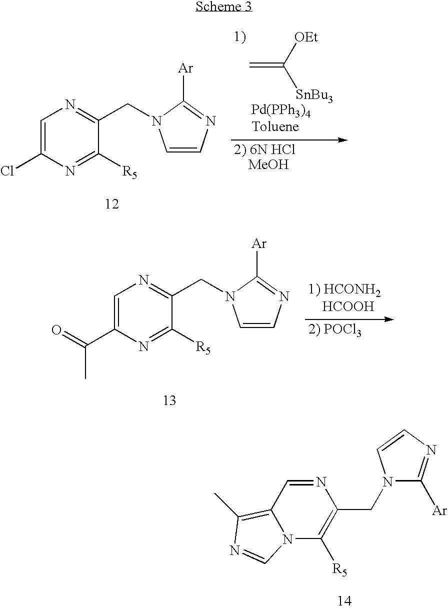

Scheme 3 illustrates the synthesis of imidazole fused pyrazines 14. 12 reacts with tributyltinvinylethylether in the presence of Pd(PPh₃)₄. Subsequent acid hydrolysis affords ketone 13. 13 reacts with formamide and formic acid, followed by POCl₃ to give product 14.

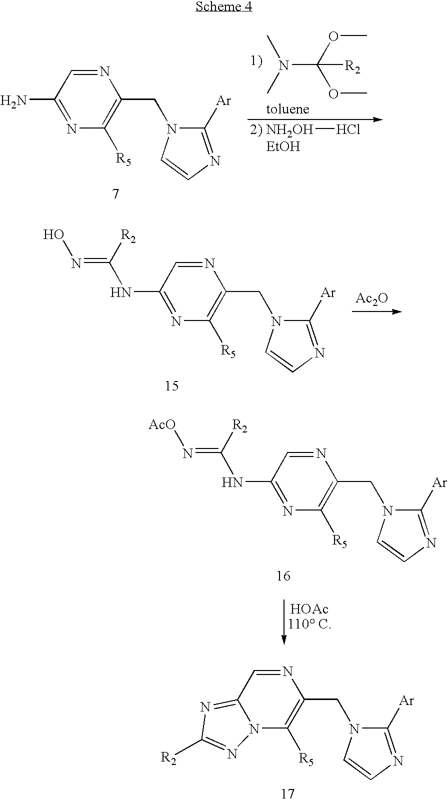

Scheme 4 illustrates the synthesis of triazole fused pyrazines 17. Reaction of 7 with an N-(1,1,-dimethoxyalkyl)-N,N-dimethylamine, followed by hydroxylamine treatment gives intermediate 15. Acetylation of 15 with acetic anhydride and subsequent cyclization in acetic acid affords product 17.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g. ³⁵S) or iodine (e.g. ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

The following Examples are offered by way of illustration and not by way of limitation. Unless otherwise specified, all reagents and solvents are of standard commercial grade and are used without further purification. Starting materials and intermediates described herein may generally be obtained from commercial sources, prepared from commercially available organic compounds or prepared using well known synthetic methods.

### EXAMPLES

Starting materials and various intermediates described in the following Examples may be obtained from commercial sources, prepared from commercially available organic compounds, or prepared using known synthetic methods. Representative examples of methods suitable for preparing intermediates of the invention are also set forth below.

In the following Examples, LC/MS conditions for the characterization of the compounds herein are:
1. Analytical HPLC/MS instrumentation: Analyses are performed using a Waters 600 series pump (Waters Corporation, Milford, Mass.), a Waters 996 Diode Array Detector and a Gilson 215 auto-sampler (Gilson Inc, Middleton, Wis.), Micromass® LCT time-of-flight electrospray ionization mass analyzer. Data are acquired using MassLynx™ 4.0 software, with OpenLynx Global Server™, OpenLynx™ and AutoLynx™ processing.
2. Analytical HPLC conditions: 4.6×50 mm, Chromolith™ SpeedROD RP-18e column (Merck KGaA, Darmstadt, Germany); UV 10 spectra/sec, 220-340 nm summed; flow rate 6.0 mL/min; injection volume 1 μl;
Gradient conditions—mobile phase A is 95% water, 5% MeOH with 0.05% TFA; mobile phase B is 95% MeOH, 5% water with 0.025% TFA, and the gradient is 0-0.5 minutes 10-100% B, hold at 100% B to 1.2 minutes, return to 10% B at 1.21 minutes inject-to-inject cycle time is 2.15 minutes.
3. Analytical MS conditions: capillary voltage 3.5 kV; cone voltage 30V; desolvation and source temperature are 350° C. and 120° C., respectively; mass range 181-750 with a scan time of 0.22 seconds and an inter scan delay of 0.05 minutes.

### Example 1

### Synthesis of 6-[2-(6-fluoro-pyridine-2-yl)-imidazol-1-ylmethyl]-5-propyl-imdazo[1,2-a]pyrazine

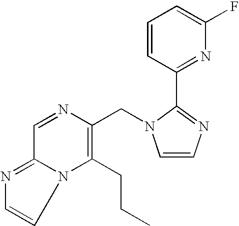 1. 5-Methyl-6-propyl-pyrazin-2-ol

This compound is prepared essentially as described by J. Am. Chem. Soc. 74:1580 (1952). The resulting mixture of two isomers is used in the next step without further purification. LC-MS: (M+1) 153.10.

2. 5-Chloro-2-methyl-3-propyl-pyrazine

The mixture of isomers (5 g) from step 1 containing 5-methyl-6-propyl-pyrazin-2-01 and POCl₃ (10 mL) is heated at 85° C. for two hours. The excess of POCl₃ is removed under vacuum, and ice water is added to the residue. The mixture is made alkaline with sat. NaHCO₃, and extracted with DCM. The organic layer is dried over MgSO₄ and the solvent is removed. The crude product is purified by passage over a silica gel column with 10:1 hexane:ethyl acetate to furnish a mixture of two isomers as a colorless oil.

3. 5-Chloro-2-methyl-3-propyl-pyrazin-1-ol

The mixture (0.9 g) from step 2 containing 5-chloro-2-methyl-3-propyl-pyrazine and mCPBA (1.7 g) in 1,2-dichloroethane (20 mL) is heated at 65° C. overnight. The mixture is cooled to room temperature, washed with sat NaHCO₃, and dried with MgSO₄, and the solvent is removed. The residue is purified using a silica gel column with 5:2 hexane:ethyl acetate to give 5-chloro-2-methyl-3-propyl-pyrazin-1-ol: ¹H NMR δ (CDCl₃) 1.01 (t, 3H, J=7.5 Hz), 1.73 (p, 2H, J=7.5 Hz), 2.44 (s, 3H), 2.78 (t, 2H, J=7.5 Hz), 8.09 (s, 1H). LC-MS (M+1): 187.06.

4. 5-Chloro-2-chloromethyl-3-propyl-pyrazine

A mixture of 5-chloro-2-methyl-3-propyl-pyrazin-1-ol (0.3 g) and POCl₃ (0.5 mL) is heated under reflux for 1 hour. The excess POCl₃ is removed under vacuum. The residue is dissolved in DCM, washed with sat. NaHCO₃, and dried with MgSO4, and the solvent is removed to give an oil, which is purified by silica gel column with 50:1 hexane:ether to furnish 5-chloro-2-chloromethyl-3-propyl-pyrazine as a colorless oil. ¹H NMR δ (CDCl₃) 1.03 (t, 3H, J=7.5 Hz), 1.81 (p, 2H, J=7.2 Hz), 2.86 (t, 2H, J=7.5 Hz), 4.70 (s, 2H), 8.38 (s, 1H). LC-MS (M+1) 205.04.
5. 2-Fluoro-6-1H-imidazol-2-yl)-pyridine

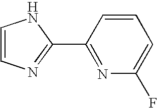

a. Preparation of 2-Fluoropyridine-6-carboxaldehyde

A solution of N-butyllithium (17.1 mL, 2.5M in hexanes) is added dropwise to a solution of diisopropylamine (6.54 mL, 1.2 equiv) in 30 mL of THF at 0° C. Stirring is continued for 15 minutes at 0° C., the reaction is then cooled to −78° C. 2-Fluoro-6-methylpyridine (4.00 mL, 38.9 mmol) is added dropwise to the cold solution. The reaction mixture is stirred at −78° C. for 1 hour and then quenched with DMF (4.52 mL, 1.5 equiv). The reaction is maintained at −78° C. for 30 minutes and then warmed to 0° C. The cold solution is added to a mixture of sodium periodate (24.9 g) in 120 mL of water at 0° C. The reaction mixture is allowed to gradually warm to room temperature over 1 hour and then stirred at room temperature for 24 hours. The reaction mixture is filtered through a plug of celite to remove the precipitate and the plug is washed with ether. The organic layer is separated, washed with aqueous sodium bicarbonate (1×40 mL), then with 0.25M KH₂PO₄ (1×40 mL) and then brine (1×40 mL). The organic solution is dried (NaSO₄) and concentrated in vacuo.

b. Preparation of 2-Fluoro-6-(1H-imidazol-2-yl)-pyridine

Methanol (12 mL) aqueous glyoxal (6.21 mL, 40 wt. % in water) is added dropwise to a solution of the crude aldehyde from step a. The solution is cooled to 0° C. and aqueous ammonium hydroxide (6.0 mL, 28 wt. % in water) is added. The reaction is allowed to warm to room temperature gradually over about an hour and then stirred another 3 hours at room temperature. Most of the methanol is removed in vacuo, the reaction mixture diluted with water (10 mL) and extracted with ethyl acetate (30 mL). The organic layer is washed with brine (20 mL), diluted with hexanes (15 mL), passed through a plug of silica gel (¼ inch deep×1¼ inch diameter), and the plug washed with more 2:1 ethyl acetate/hexanes (20 mL). The combined eluents are concentrated in vacuo to yield crude 2-fluoro-6-(1H-imidazol-2-yl)-pyridine.

6. 5-Chloro-2-[2-(6-fluoro-pyridin-2-yl)-imidazol-1-ylmethyl]-3-propyl-pyrazine

A mixture of 5-chloro-2-chloromethyl-3-propyl-pyrazine (0.108 g), 2-fluoro-6-(1H-imidazol-2-yl)-pyridine (0.086 g), and K₂CO₃ (0.095 g) in DMF (1 mL) is stirred at room temperature overnight. Water (5 mL) is added. The mixture is extracted with ethyl acetate (15 mL×3), dried, and solvent evaporated to give the crude product, which is purified by silica column with 5% methanol in DCM to give the title product ¹H NMR δ (CDCl₃) 0.99 (t, 3H, J=7.5 Hz), 1.76 (p, 2H, J=7.2 Hz), 1.91 (t, 2H, J=7.5 Hz), 6.04 (s, 2H), 6.80 (dd, 1H, J=2.7, 0.6 Hz), 7.09 (d, 1H, J=1.2 Hz), 7.21 (d, 1H, J=1.2 Hz), 7.82 (q, 1H, J=7.5 Hz), 8.14 (dd, 1H, J=2.7, 0.6 Hz), 8.24 (s, 1H). LC-MS (M+1) 332.07.
7. Benzhydrylidene-{5-[2-(6-fluoro-pyridin-2-yl)-imidazol-1-ylmethyl]-6-propyl-pyrazin-2-yl}-amine

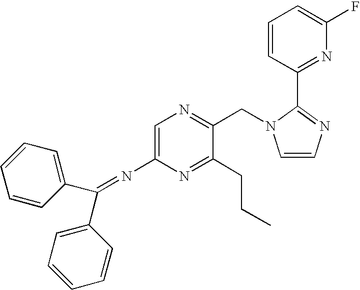

A round-bottom sealed tube is purged with nitrogen and charged with Pd(OAc)₂ (14 mg, 5%), BINAP (43 mg, 5%), and dry THF. The mixture is flushed with N₂ for approximately 5 minutes. While stirring, 5-chloro-2-[2-(6-fluoro-pyridin-2-yl)-imidazol-1-ylmethyl]-3-propyl-pyrazine (0.21 g), benzophenone imine (0.13 g) and Cs₂CO₃ (0.42 g) are added, and the mixture is heated at 90° C. until the starting material has been consumed. The mixture is cooled to room temperature. THF is removed and ethyl acetate (40 ml) is added. The mixture is washed with water (10 ml), brine (10 ml) and dried, and solvent is removed to give the crude product. The crude is purified by silica column with 2:1 ethyl acetate:hexane to give the title compound: ¹H NMR δ (CDCl₃) 0.79 (t, 3H, J=7.5 Hz), 1.52 (p, 2H, J=7.2 Hz), 2.74 (t, 2H, J=7.5 Hz), 5.93 (s, 2H), 6.80 (dd, 1H, J=2.7, 0.6 Hz), 6.91 (s, 1H), 7.05-7.30 (m, 6H), 7.34-7.49 (m, 3H), 7.63 (s, 1H), 7.75-7.85 (m, 3H), 8.07-8.11 (m, 1H). LC-MS (M+1) 477.15.
8. 5-[2-(6-Fluoro-pyridin-2-yl)-imidazol-1-ylmethyl]-6-propyl-pyrazin-2-ylamine

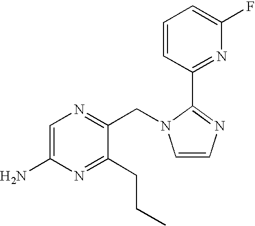

Benzhydrylidene-{5-[2-(6-fluoro-pyridin-2-yl)-imidazol-1-ylmethyl]-6-propyl-pyrazin-2-yl}-amine (0.1 g) is dissolved in THF (15 mL) at room temperature. 10 mL of 5% HCl (aq.) is added, and the mixture is stirred at room temperature for 30 minutes. THF is removed and the mixture is neutralized with sat NaHCO₃. The mixture is extracted with chloroform (30 mL×3). The organic phase is dried over MgSO₄. The solvent is removed to give a white solid, which is washed with ether to give the title product LC-MS (M+1) 313.14.
9. 6-[2-(6-Fluoro-pyridin-2-yl)-imidazol-1-ylmethyl]-5-propyl-imidazo[1,2-a]pyrazine

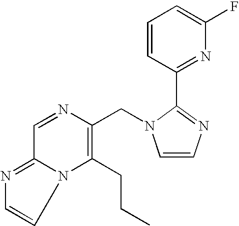

A mixture of 5-[2-(6-fluoro-pyridin-2-yl)-imidazol-1-ylmethyl]-6-propyl-pyrazin-2-ylamine (20 mg) and 50% chloroacetaldehyde in water (0.2 mL) in DMF (5 mL) is heated at 70° C. overnight. Ethyl acetate (20 mL) is added. The mixture is washed with sat. NaHCO₃, and dried. PTLC separation with 5% methanol in DCM gives the title product. ¹H NMR δ (CDCl₃) 0.97 (t, 3H, J=7.5 Hz), 1.63 (p, 2H, J=7.2 Hz), 3.17 (t, 2H, J=7.5 Hz), 6.14 (s, 2H), 6.87 (dd, 1H, J=2.7, 0.6 Hz), 7.13 (d, 1H, J=1.2 Hz), 7.28 (d, 1H, J=1.2 Hz), 7.66 (s, 1H), 7.83 (s, 1H), 7.86 (q, 1H, J=7.5 Hz), 8.15 (dd, 1H, J=2.7, 0.6 Hz), 8.98 (s, 1H). LC-MS (M+1) 337.14.

### Example 2

### Synthesis of 5-propyl-6-(2-pyridi-2-yl-imidazol-1-ylmethyl)-imidazo[1,2-a]pyrazine

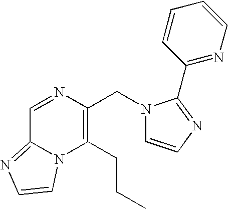

This compound is prepared as described in Example 1, with readily apparent modifications. ¹H NMR δ (CDCl₃) 0.91 (t, 3H, J=7.5 Hz), 1.54 (p, 2H, J=7.2 Hz), 3.08 (t, 2H, J=7.5 Hz), 6.24 (s, 2H), 7.11 (s, 1H), 7.24-7.28 (m, 2H), 7.62 (s, 1H), 7.70-7.85 (m, 2H), 8.26 (d, 1H, J=8.1 Hz), 8.59 (s, 1H), 8.99 (s, 1H). LC-MS: (M+1) 319.15.

### Example 3

### Synthesis of 6-[2-(3-fluoro-pyridin-2-yl)-imidazol-2-ylmethyl-5-propyl-imidazo[1,2-a]pyrazine

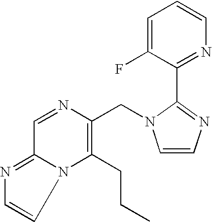 This compound is prepared as described in Example 1, with readily apparent modifications. ¹H NMR δ (CDCl₃) 0.92 (t, 3H, J=7.5 Hz), 1.54 (p, 2H, J=7.2 Hz), 2.97 (t, 2H, J=7.5 Hz), 5.87 (s, 2H), 7.18-7.33 (m, 2H), 7.35-7.40 (m, 1H), 7.54-7.64 (m, 2H, 7.85 (s, 1H), 8.50 (s, 1H), 8.98 (s, 1H). LC-MS: (M+1) 337.11.

### Example 4

### Synthesis of 6-[2-(6-fluoro-pyridin-2-ylmethyl]-1-methyl-5-propyl-imidazo[1,5-a]pyrazine

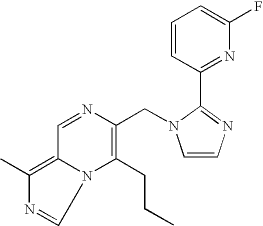 1. 1-{5-[2-(6-Fluoro-pyridin-2-yl)-imidazol-1-ylmethyl]-6-propyl-pyrazin-2-yl}-ethanone

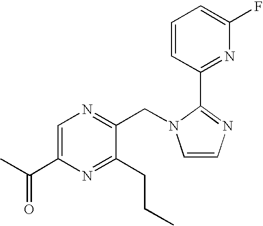

Tributyltinvinylethylether (0.40 g) and Pd(Ph₃P)₂C₂ (40 mg) are added to a solution of 5-chloro-2-[2-(6-fluoro-pyridin-2-yl)-imidazol-1-ylmethyl]-3-propyl-pyrazine (0.24 g) in toluene (30 mL). The mixture is degassed for 10 minutes, and then heated at 130° C. overnight. The solvent is removed under vacuum, and the residue is dissolved in methanol (15 mL). 6N HCl (20 mL) is added, and the mixture is stirred at room temperature for 5 hours. The solvent is removed, neutralized with saturated NaHCO₃, and extracted with ethyl acetate. The organic layers are dried and solvent evaporated to give the crude product, which is purified by PTLC with 5% methanol in DCM to give the title product ¹H NMR δ (CDCl₃) 1.04 (t, 3H, J=7.5 Hz), 1.88 (p, 2H, J=7.2 Hz), 2.69 (s, 3H), 3.00 (t, 2H, J=7.5 Hz), 6.08 (s, 2H), 6.76 (dd, 1H, J=7.8, 2.7 Hz), 7.10 (s, 1H), 7.23 (s, 1H), 7.80 (q, 1H, J=8.1 Hz), 8.13 (dd, 1H, J=7.8, 2.1 Hz), 8.84 (s, 1H). LC-MS (M+1) 386.20.
2. N-(1-{5-[2-(6-fluoro-pyridin-2-yl)-imidazol-1-ylmethyl]-6-propyl-pyrazin-2-yl}-ethyl)-formamide

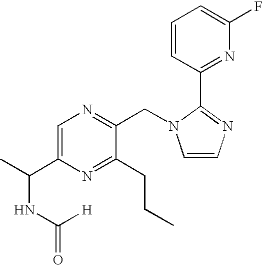

To 0.6 g of formamide at 160-180° C. is added 1-{5-[2-(6-fluoro-pyridin-2-yl)-imidazol-1-ylmethyl]-6-propyl-pyrazin-2-yl}-ethanone (0.12 g) and formic acid (0.050 g) in 0.5 g of formamide. The mixture is heated at 160-180° C. for an additional 1.5 hours. During this period, formic acid (0.050 g) is added. The mixture is cooled to room temperature and poured into water (10 mL), and the solution is made alkaline to at least pH 11 with concentrated sodium hydroxide. The solution is extracted with ethyl acetate. The organic layers are dried over MgSO₄, and solvent evaporated to give the crude product, which is purified by TLC with ethyl acetate to give the title product ¹H NMR δ (CDCl₃) 0.98 (t, 3H, J=7.5 Hz), 1.45 (d, 3H, J=6.9 Hz), 1.72 (p, 2H, J=7.2 Hz), 2.90 (t, 2H, J=7.5 Hz), 5.25 (p, 1H, J=6.9 Hz), 6.05(q, 2H, J=10.3 Hz), 6.76-6.83(m, 2), 7.08 (s, 1H), 7.19 (s, 1H), 7.81 (q, 1H, J=8.1 Hz), 8.15(dd, 1H, J=7.8, 2.1 Hz), 8.19 (s, 1H). LC-MS: (M+1) 369.16.
3. 6-[2-(6-Fluoro-pyridin-2-ylmethyl]-1-methyl-5-propyl-imidazo[1,5-a]pyrazine

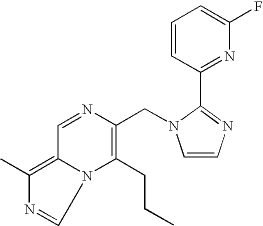

A mixture of N-(1-{5-[2-(6-fluoro-pyridin-2-yl)-imidazol-1-ylmethyl]-6-propyl-pyrazin-2-yl}-ethyl)-formamide (70 mg) and POCl₃ (3 ml) is heated at reflux for 3 hours. Excess POCl₃ is removed, ether acetate (10 mL) is added, and the mixture is washed with saturated NaHCO₃ (5 mL) and brine (5 mL), and dried over MgSO₄. After evaporation of the solvent, the residue is purified by PTLC with 7% methanol in DCM to give the title product ¹H NMR δ (CDCl₃) 0.97 (t, 3H, J=7.5 Hz), 1.26 (s, 3H), 1.65 (p, 2H, J=7.2 Hz), 2.61 (s, 3H), 3.09 (t, 2H, J=7.5 Hz), 6.03 (s, 2H), 6.92 (d, 1H, J=2.7 Hz), 7.20 (s, 1H), 7.36 (s, 1H), 7.92 (q, 1H, J=7.5 Hz), 8.07 (s, 1H), 8.30 (s, 1H), 8.75 (s, 1H). LC-MS: (M+1) 351.14.

### Example 5

### Synthesis of 6-[2-(3-fluoro-pyridin-2-yl)-imidazol-1-ylmethyl]-1 methyl-5-propyl-imidazo[1,5-a]pyrazine

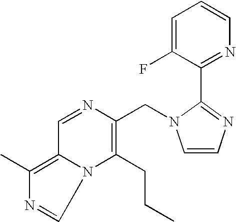 This compound is prepared as described in Example 4, with readily apparent modifications. ¹H NMR δ (CDCl₃) 0.91 (t, 3H, J=7.5 Hz), 1.55 (p, 2H, J=7.2 Hz), 2.60 (s, 3H), 2.87 (t, 2H, J=7.5 Hz), 5.74 (s, 2H), 7.21 (d, 1H, J=2.7 Hz), 7.20 (s, 1H), 7.36 (m, 1H), 7.57(t, 1H, J=9.6 Hz), 8.01 (s, 1H), 8.48 (d, 1H, J=4.5 Hz), 8.75 (s, 1H). LC-MS: (M+1) 351.14.

### Example 6

### Synthesis of 5-propyl-6-(2-pyridin-2-yl-imidazol-1-ylmethyl)-[1,2,4]triazolo[4,3-a]pyrazine

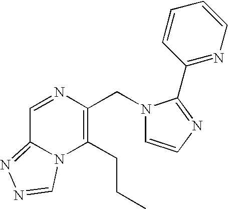 1. 5-Chloro-3-propyl-2-(2-pyridin-2-yl-imidazol-1-ylmethyl)-pyrazine

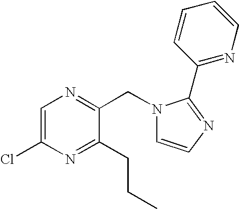

A mixture of 5-chloro-2-chloromethyl-3-propyl-pyrazine (0.35 g), 2-(1H-imidazol-2-yl)-pyridine (0.25 g), and K₂CO₃ (0.28 g) in DMF (10 mL) is stirred at room temperature overnight. Water (15 mL) is added. The mixture is extracted with DCM (15 mL×3). The organic layers are dried and solvent evaporated to give a residue, which is purified by silica gel column with 7.5% methanol in DCM to give the title product: ¹H NMR δ (CDCl₃) 0.95 (t, 3H, J=7.5 Hz), 1.70 (p, 21, J=7.2 Hz), 1.85 (t, 2H, J=7.5 Hz), 6.11 (s, 2H), 7.07 (d, 1H, J=1.2 Hz), 7.14-7.21 (m, 2H), 7.70-7.77 (m, 1H), 8.23 (d, 1H, J=6.0 Hz), 8.27(s, 1H), 8.39-8.43 (m, 1H). LC-MS: (M+1) 314.10.
2. 6-Propyl-5-(2-pyridin-2-yl-imidazol-1-ylmethyl)-pyrazin-2-yl]-hydrazine

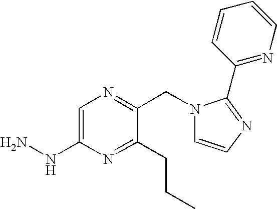

A mixture of 5-chloro-3-propyl-2-(2-pyridin-2-yl-imidazol-1-ylmethyl)-pyrazine (0.103 g) and hydrazine hydrate (0.2 mL) in ethanol (10 mL) is heated at 110° C. in a sealed tube overnight. The solvent is removed under vacuum to give a solid, which is washed with ethyl acetate and dried to give the title product: ¹H NMR δ (CDCl₃) 0.59 (t, 3H, J=7.5 Hz), 1.28 (p, 2H, J=7.2 Hz), 2.34 (t, 2H, J=7.5 Hz), 3.31 (m, 3H), 5.74 (s, 2H), 6.83 (d, 1H, J=4.5 Hz), 7.0.5-7.28 (m, 1H), 7.26 (s, 1H), 7.63 (t, 1H, J=4.5 Hz), 7.72 (s, 1H), 7.81 (d, 1H, J=7.2 Hz), 8.40 (d, 1H, J=4.8 Hz). LC-MS: (M+1) 310.13.
3. 5-Propyl-6-(2-pyridin-2-yl-imidazol-1-ylmethyl)-[1,2,4]triazolo[4,3-a]pyrazine

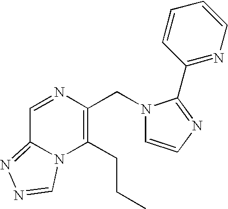

A mixture of 6-propyl-5-(2-pyridin-2-yl-imidazol-1-ylmethyl)-pyrazin-2-yl]-hydrazine (26 mg) and formic acid (2 m) is heated at 110° C. overnight. The excess formic acid is removed, and methylene chloride (10 mL) is added. The mixture is washed with sat. NaHCO₃, dried, and solvent evaporated to give a residue, which is purified by PTLC with 5% methanol in methylene chloride to give the title product: ¹H NMR δ (CDCl₃) 0.95 (t, 3H, J=7.5 Hz), 1.61 (p, 2H, J=7.2 Hz), 3.18 (t, 2H, J=7.5 Hz), 6.24 (s, 2H), 7.14 (s, 1H), 7.23-7.28 (m, 2H), 7.78 (t, 1H, J=5.7 Hz), 8.25 (d, 1H, J=6.0 Hz), 8.56 (d, 1H, J=3.6 Hz), 8.86 (s, 1H), 9.24 (s, 1H). LC-MS: (M+1) 320.12.

### Example 7

### Synthesis of 3-methyl-5-propyl-6-(2-pyridin-2-yl-imidazol-1-ylmethyl)-[1,2,4]triazolo[4,3-a]pyrazine

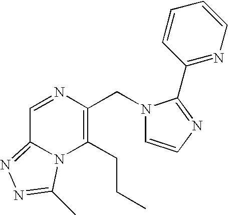

This compound is prepared as described in Example 6, with readily apparent modifications. ¹H NMR δ (CDCl₃) 0.95 (t, 3H, J=7.5 Hz), 1.54 (p, 2H, J=7.2 Hz), 2.98 (s, 3H), 3.22 (t, 2H, J=7.5 Hz), 6.23 (s, 2H), 7.14 (s, 1H), 7.24-7.28 (m, 2H), 7.79 (t, 1H, J=5.7 Hz), 8.26 (d, 1H, J=5.4 Hz), 8.57 (s, 1H), 9.13 (s, 1H). LC-MS: (M+1) 334.13.

### Example 8

### Synthesis of 6-{[2-(3-fluoropyridin-2-yl)-1H-imidazol-1-yl]methyl}-5-propyl[1,2,4]triazolo[1,5-a]pyrazine

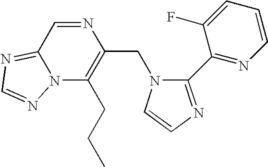 1. 5-{[2-(3-fluoropyridin-2-yl)-1H-imidazol-1-yl]methyl}-6-propylpyrazin-2-amine

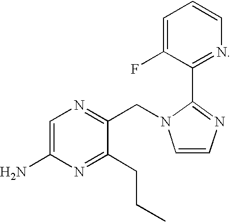

This compound is prepared as described in Example 1, steps 1-8, with readily apparent modifications.
2. N-5-{[2-(3-fluoropyridin-2-yl)-1H-imidazol-1-yl]methyl}-6-propylpyrazin-2-yl-N′-hydroxy-imidoformamide

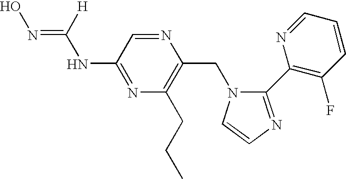

A solution of 5-{[2-(3-fluoropyridin-2-yl)-1H-imidazol-1-yl]methyl}-6-propylpyrazin-2-amine (210 mg, 0.67 mmol) and N-(dimethyoxymethyl)-N,N-dimethylamine (0.67 mmol) in toluene (3 ml) is refluxed for 3 hours. The solvent is removed in vacuo and the resulting yellow oil is dissolved in EtOH (6 ml) and to it is added NH₂OH—HCl (76 mg, 1.1 mmol). The mixture is stirred at room temperature overnight The solvent is removed in vacuo and the residue is partitioned between saturated aqueous NaHCO₃ solution (5 ml) and EtOAc (10 ml). The layers are separated and the aqueous layer is extracted with EtOAc (2×10 ml). The combined extracts are washed with brine (8 ml), dried (Na₂SO₄) and evaporated. Preparative TLC separation of the residue with 5% MeOH in CH₂CH₂ gives the titled compound as a yellow solid.

A mixture of N-5-{[2-(3-fluoropyridin-2-yl)-1H-imidazol-1-yl]methyl}-6-propylpyrazin-2-yl-N′-hydroxyimidoformamide hydroxyethanimidamide (0.7 mmol) and acetic anhydride (2 ml) is stirred at room temperature for 4 hours. The solvent is removed in vacuo and the residue is partitioned between saturated aqueous NaHCO₃ solution (10 ml) and EtOAc (20 ml). The layers are separated and the aqueous layer is extracted with EtOAc (3×20 ml). The combined extracts are washed with brine (15 ml), dried (Na₂SO₄) and evaporated. The yellow oil residue is used in the next step without further purification.
3. 6-{[2-(3-fluoropyridin-2-yl)-1H-imidazol-1-yl]methyl}-5-propyl[1,2,4]triazolo[1,5-a]pyrazine

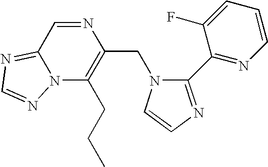

A mixture of N-5-{[2-(3-fluoropyridin-2-yl)-1H-imidazol-1-yl]methyl}-6-propylpyrazin-2-yl-N′-hydroxyimidoformamide hydroxyethanimidamide (0.7 mmol) and acetic anhydride (2 ml) is stirred at room temperature for 4 hours. The solvent is removed in vacuo and the residue is partitioned between saturated aqueous NaHCO₃ solution (10 ml) and EtOAc (20 ml). The layers are separated and the aqueous layer is extracted with EtOAc (3×20 ml). The combined extracts are washed with brine (15 ml), dried (Na₂SO₄) and evaporated. The resulting yellow oil residue is dissolved in HOAc (6 ml) and the mixture is heated at 110° C. overnight. The solvent is removed in vacuo and the residue is partitioned between saturated aqueous NaHCO₃ solution (5 ml) and EtOAc (10 ml). The layers are separated and the aqueous layer is extracted with EtOAc (2×10 ml). The combined extracts are washed with brine (8 ml), dried (Na₂SO₄) and evaporated. Preparative TLC separation of the residue with 5% MeOH in CH₂CH₂ gives the titled compound as a pale yellow solid. ¹H NMR δ (CDCl₃) 9.13 (s, 1H), 8.45-8.47 (m, 1H), 8.46 (s, 1H), 7.55-7.60 (m, 1H), 7.32-7.36 (m, 1H), 7.24 (s, 1H), 7.22 (s, 1H), 5.96 (s, 2H), 3.18-3.22 (m, 2H), 1.59-1.68 (m, 2H), 0.94 (t, 3H).

### Example 9

### Synthesis of 6-{[2-(3-fluoropyridin-2-yl)-1H-imidazol-1-yl]methyl}-2-methyl-5-propyl[1,2,4]triazolo[1,5-a]pyrazine

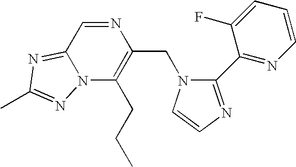

This compound is prepared as described in Example 8, with readily apparent modifications. ¹H NMR (8, CDCl₃) 8.99 (s, 1H), 8.47-8.48 (m, 1H), 7.56-7.61 (m, 1H), 7.33-7.37 (m, 1H), 7.24 (s, 1H), 7.21 (s, 1H), 5.93 (s, 2H), 3.13-3.17 (m, 2H), 2.65 (s, 3H), 1.60-1.66 (m, 2H), 0.93 (t, 3H).

### Example 10

### Ligand Binding Assay

The high affinity of preferred compounds of this invention for the benzodiazepine site of the GABA_{A} receptor is confirmed using a binding assay essentially described by Thomas and Tallman (J. Bio. Chem. (1981) 156:9838-9842, and J. Neurosci. (1983) 3:433440).

Rat cortical tissue is dissected and homogenized in 25 volumes (w/v) of Buffer A (0.05 M Tris HCl buffer, pH 7.4 at 4° C.). The tissue homogenate is centrifuged in the cold (4° C.) at 20,000×g for 20 minutes. The supernatant is decanted, the pellet rehomogenized in the same volume of buffer, and centrifuged again at 20,000×g. The supernatant of this centrifugation step is decanted and the pellet stored at −20° C. overnight. The pellet is then thawed and resuspended in 25 volumes of Buffer A (original wt/vol), centrifuged at 20,000×g and the supernatant decanted. This wash step is repeated once. The pellet is finally resuspended in 50 volumes of Buffer A.

Incubations contain 100 μl of tissue homogenate, 100 μl of radioligand, (0.5 nM ³H-RO15-1788 [³H-Flumazenil], specific activity 80 Ci/mmol), and test compound or control (see below), and are brought to a total volume of 500 μl with Buffer A. Incubations are carried out for 30 minutes at 4° C. and then rapidly filtered through Whatman GFB filters to separate free and bound ligand. Filters are washed twice with fresh Buffer A and counted in a liquid scintillation counter. Nonspecific binding (control) is determined by displacement of ³H RO15-1788 with 10 μM Diazepam (Research Biochemicals International, Natick, Mass.). Data are collected in triplicate, averaged, and percent inhibition of total specific binding (Total Specific Binding=Total−Nonspecific) is calculated for each compound.

A competition binding curve is obtained with up to 11 points spanning the compound concentration range from 10⁻¹²M to 10⁻⁵M obtained per curve by the method described above for determining percent inhibition. Kᵢ values are calculated according the Cheng-Prussof equation Preferred compounds of the invention exhibit Kᵢ values of less than 100 nM and more preferred compounds of the invention exhibit Kᵢ values of less than 10 nM.

### Example 11

### Electrophysiology

The following assay is used to determine if a compound of the invention alters the electrical properties of a cell and if it acts as an agonist, an antagonist, or an inverse agonist at the benzodiazepine site of the GABA_{A} receptor.

Assays are carried out essentially as described in White and Gurley (NeuroReport 6:1313-1316, 1995) and White, Gurley, Hartnett, Stirling, and Gregory (Receptors and Channels 3:1-5, 1995) with modifications. Electrophysiological recordings are carried out using the two electrode voltage-clamp technique at a membrane holding potential of −70 mV. Xenopus laevis oocytes are enzymatically isolated and injected with non-polyadenylated cRNA mixed in a ratio of 4:1:4 for α, β and γ subunits, respectively. Of the nine combinations of α, β and γ subunits described in the White et al. publications, preferred combinations are α₁β₂γ₂, α₂β₃γ₂, α₃β₃γ₂, and α₅β₃γ₂. Preferably all of the subunit cRNAs in each combination are human clones or all are rat clones. Each of these cloned subunits is described in GENBANK, e.g., human α₁, GENBANK accession no. X14766, human α₂, GENBANK accession no. A28100; human α₃, GENBANK accession no. A28102; human α₅, GENBANK accession no. A28104; human β₂, GENBANK accession no. M82919; human β₃, GENBANK accession no. Z₂₀₁₃₆; human γ₂, GENBANK accession no. X15376; rat α₁, GENBANK accession no. L08490, rat α₂, GENBANK accession no. L08491; rat α₃, GENBANK accession no. L08492; rat α₅, GENBANK accession no. L08494; rat β₂, GENBANK accession no. X15467; rat β₃, GENBANK accession no. X15468; and rat γ₂, GENBANK accession no. L08497. For each subunit combination, sufficient message for each constituent subunit is injected to provide current amplitudes of >10 nA when 1 μM GABA is applied.

Compounds are evaluated against a GABA concentration that evokes <10% of the maximal evocable GABA current (e.g., 1 μm-9M). Each oocyte is exposed to increasing concentrations of a compound being evaluated (test compound) in order to evaluate a concentration/effect relationship. Test compound efficacy is calculated as a percent-change in current amplitude: 100*((Ic/I)−1), where Ic is the GABA evoked current amplitude observed in the presence of test compound and I is the GABA evoked current amplitude observed in the absence of the test compound.

Specificity of a test compound for the benzodiazepine site is determined following completion of a concentration/effect curve. After washing the oocyte sufficiently to remove previously applied test compound, the oocyte is exposed to GABA+1 μM RO15-1788, followed by exposure to GABA+1 μM RO15-1788+test compound. Percent change due to addition of compound is calculated as described above. Any percent change observed in the presence of RO15-1788 is subtracted from the percent changes in current amplitude observed in the absence of 1 μM RO15-1788. These net values are used for the calculation of average efficacy and EC₅₀ values by standard methods. To evaluate average efficacy and EC₅₀ values, the concentration/effect data are averaged across cells and fit to the logistic equation.

### Example 12

### MDCK Toxicity Assay This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet Confluent MDCK cells are trypsinized, harvested, and diluted to a concentration of 0.1×10⁶ cells/ml with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The PACKARD, (Meriden, Conn.) ATP-LITE-M Luminescent ATP detection kit, product no. 6016941, is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 ml of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A compound of the Formula: 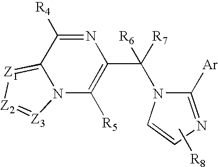
or a pharmaceutically acceptable form thereof, wherein:
Z₁ is nitrogen or CR₁; Z₂ is nitrogen or CR₂; Z₃ is nitrogen or CR₃; and at least one, but no more than two of Z₁, Z₂ and Z₃ are nitrogen;
Ar represents phenyl, naphthyl or 5- to 10-membered heteroaryl, each of which is substituted with from 0 to 4 substituents independently chosen from halogen, hydroxy, nitro, cyano, amino, C₁-C₈alkyl, C₁-C₈alkenyl, C₁-C₈alkynyl, C₁-C₈alkoxy, C₃-C₇cycloalkyl, (C₃-C₇cycloalkyl)C₀-C₄alkyl, (C₃-C₇cycloalkyl)C₁-C₄alkoxy, C₁-C₈alkyl ether, C₁-C₈alkanone, C₁-C₈alkanoyl, 3- to 7-membered heterocycloalkyl, C₁-C₈haloalkyl, C₁-C₈haloalkoxy, oxo, C₁-C₈hydroxyalkyl, C₁-C₈aminoalkyl and mono- and di-(C₁-C₈alkyl)amino(C₀-C₈alkyl);
R₁, R₂, R₃, and R₄ are each independently selected from:
(a) hydrogen, halogen, nitro and cyano; and
(b) groups of the formula: 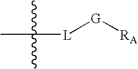
wherein:
L is a single covalent bond or C₁-C₈alkyl;
G is a single covalent bond, —N(R_{B})—, —O—, —C(═O)—, —C(═O)O—, —C(═O)N(R_{B})—, —N(R_{B})C(═O)—, —S(O)ₘ, —CH₂C(═O)—, —S(O)ₘN(R_{B})— or —N(R_{B})S(O)ₘ—; wherein m is 0, 1 or 2; and
R_{A} and each R_{B} are independently selected from:
(i) hydrogen; and
(ii) C₁-C₈alkyl, C₂-C₈alkenyl, C₂-C₈alkynyl, (C₃-C₈cycloalkyl)C₀-C₄alkyl, (3- to 6-membered heterocycloalkyl)C₀-C₄alkyl, (aryl)C₀-C₂alkyl or (heteroaryl)C₀-C₂alkyl, each of which is substituted with from 0 to 4 substituents independently selected from halogen, hydroxy, nitro, cyano, amino, C₁-C₄alkyl, C₁-C₄alkoxy, C₁-C₄alkanoyl, mono- and di(C₁-C₄alkyl)amino, C₁-C₄haloalkyl and C₁-C₄haloalkoxy;
R₅ is C₁-C₆alkyl, C₂-C₆alkenyl, C₁-C₄alkoxy, or mono- or di-(C₁-C₄alkyl)amino, each of which is substituted with from 0 to 5 substituents independently chosen from halogen, hydroxy, nitro, cyano, amino, C₁-C₄alkoxy, C₁-C₂haloalkyl, C₁-C₂haloalkoxy, mono- and di-C₁-C₄alkylamino, C₃-C₈cycloalkyl, phenylC₀-C₄alkyl and phenylC₁-C₄alkoxy;
R₆ and R₇ are independently hydrogen, halogen, methyl or ethyl; and
R₈ represents 0, 1 or 2 substituents independently chosen from halogen, hydroxy, nitro, cyano, amino, C₁-C₄alkyl, C₁-C₄alkoxy, mono- and di-(C₁-C₄alkyl)amino, C₃-C₇cycloalkyl, C₁-C₂haloalkyl and C₁-C₂haloalkoxy.

2. A compound or pharmaceutically acceptable form thereof according to claim 1, wherein R₈ represents 0 or 1 substituent selected from halogen, C₁-C₂alkyl and C₁-C₂alkoxy.

3-4. (canceled)

5. A compound or pharmaceutically acceptable form thereof according to claim 1, wherein Ar represents phenyl, pyridyl, thiazolyl, thienyl, triazolopyridyl, or pyridizinyl, each of which is substituted with from 0 to 3 substituents independently selected from chloro, fluoro, hydroxy, cyano, amino, C₁-C₄alkyl, C₁-C₄alkoxy, C₁-C₂alkylamino, C₁-C₂haloalkyl and C₁-C₂haloalkoxy.

6. A compound or pharmaceutically acceptable form thereof according to claim 5, wherein Ar represents phenyl, 2-pyridyl, 1,3-thiazol-2-yl, 2-thienyl, [1,2,4]triazolo[4,3-a]pyridin-5-yl or 3-pyridizinyl, each of which is substituted with from 0 to 3 substituents independently selected from fluoro, chloro, hydroxy, C₁-C₂alkyl, cyano, and C₁-C₂alkoxy.

7. A compound or pharmaceutically acceptable form thereof according to claim 5, wherein Ar represents pyridin-2-yl, 2,6-difluorophenyl, 2,5-difluorophenyl, 3-fluorophenyl, 3-methyl-[1,2,4]triazolo[4,3-a]pyridin-5-yl, 3-fluoropyridin-2-yl or 6-fluoro-pyridin-2-yl.

8. (canceled)

9. A compound or pharmaceutically acceptable form thereof according to claim 1 wherein R₁, R₂, R₃, and R₄ are independently selected from hydrogen, hydroxy, halogen, cyano, C₁-C₆alkyl, C₁-C₆alkoxy, C₃-C₇cycloalkyl, C₁-C₂alkoxyC₁-C₄alkyl, C₁-C₄hydroxyalkyl, C₁-C₂haloalkyl, C₁-C₂haloalkoxy, C₁-C₄carboxylate, mono- and di-(C₁-C₄alkyl)amino, phenylC₀-C₁alkyl, pyridylC₀-C₁alkyl and (4- to 7-membered heterocycloalkyl)C₀-C₁ alkyl.

10. A compound or pharmaceutically acceptable form thereof according to claim 9, wherein R₁ and R₄ are independently chosen from hydrogen, methyl and ethyl.

11. A compound or pharmaceutically acceptable form thereof according to claim 9, wherein Z₁ is nitrogen, Z₂ is CR₂ and Z₃ is CR₃.

12. A compound or pharmaceutically acceptable form thereof according to claim 11, wherein R₂, R₃ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenylC₀-C₁alkyl, pyridylC₀-C₁alkyl and (4- to 7-membered heterocycloalkyl)C₀-C₁alkyl.

13. A compound or pharmaceutically acceptable form thereof according to claim 9, wherein Z₁ is CR₁, Z₂ is nitrogen and Z₃ is CR₃.

14. A compound or pharmaceutically acceptable form thereof according to claim 13, wherein R₁, R₃ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenylC₀-C₁alkyl, pyridylC₀-C₁alkyl and (4- to 7-membered heterocycloalkyl)C₀-C₁alkyl.

15. A compound or pharmaceutically acceptable form thereof according to claim 9, wherein Z₁ and Z₂ are nitrogen and Z₃ is CR₃.

16. A compound or pharmaceutically acceptable form thereof according to claim 15, wherein R₃ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenylC₀-C₁alkyl, pyridylC₀-C₁alkyl and (4- to 7-membered heterocycloalkyl)C₀-C₁alkyl.

17. A compound or pharmaceutically acceptable form thereof according to claim 9, wherein Z₁ and Z₃ are nitrogen and Z₂ is CR₂.

18. A compound or pharmaceutically acceptable form thereof according to claim 17, wherein R₂ and R₄ are independently chosen from hydrogen, halogen, C₁-C₄alkyl and C₁-C₄alkoxy, C₃-C₇cycloalkyl, C₁-C₂alkoxyC₁-C₂alkyl, C₁-C₂hydroxyalkyl, fluoromethyl, difluoromethyl, trifluoromethyl, phenylC₀-C₁alkyl, pyridylC₀-C₁alkyl and (4- to 7-membered heterocycloalkyl)C₀-C₁alkyl.

19. A compound or pharmaceutically acceptable form thereof according to claim 1 wherein R₆ and R₇ are both hydrogen.

20. (canceled)

21. A compound or pharmaceutically acceptable form thereof according to claim 1 wherein R₅ is ethyl, propyl, butyl, ethoxy or methoxymethyl.

22. A compound or pharmaceutically acceptable form thereof according to claim 1, wherein the compound is chosen from:
6-[2-(6-fluoro-pyridin-2-yl)-imidazol-1-ylmethyl]-5-propyl-imidazo[1,2-a]pyrazine;
5-propyl-6-(2-pyridi-2-yl-imidazol-1-ylmethyl)-imidazo[1,2-a]pyrazine;
6-[2-(3-fluoro-pyridin-2-yl)-imidazol-2-ylmethyl]-5-propyl-imidazo[1,2-a]pyrazine;
6-[2-(6-fluoro-pyridin-2-ylmethyl]-1-methyl-5-propyl-imidazo[1,5-a]pyrazine;
6-[2-(3-fluoro-pyridin-2-yl)-imidazol-1-ylmethyl]-1-methyl-5-propyl-imidazo[1,5-a]pyrazine;
5-propyl-6-(2-pyridin-2-yl-imidazol-1-ylmethyl)-[1,2,4]triazolo[4,3-a]pyrazine;
3-methyl-5-propyl-6-(2-pyridin-2-yl-imidazol-1-ylmethyl)-[1,2,4]triazolo[4,3-a]pyrazine;
3-methyl-6-[2-(3-methyl-[1,2,4]triazolo[4,3-a]pyridin-5-yl)-imidazol-1-ylmethyl]-5-propyl-[1,2,4]triazolo[4,3-a]pyrazine;
6-{[2-(3-fluoropyridin-2-yl)-1H-imidazol-1-yl]methyl}-5-propyl[1,2,4]triazolo[1,5-a]pyrazine; and
6-{[2-(3-fluoropyridin-2-yl)-1H-imidazol-1-yl]methyl}-2-methyl-5-propyl[1,2,4]triazolo[1,5-a]pyrazine.

23-25. (canceled)

26. A pharmaceutical composition comprising a compound or pharmaceutically acceptable form thereof according to claim 1 in combination with a pharmaceutically acceptable carrier or excipient.

27. A pharmaceutical composition according to claim 26, wherein the pharmaceutical composition is formulated as an injectible fluid, an aerosol, a cream, a gel, a pill, a capsule, a syrup, or a transdermal patch.

28. A method for the treatment of anxiety, depression, or a sleep disorder, comprising administering to a patient in need of such treatment a GABA_{A} receptor modulatory amount of a compound or pharmaceutically acceptable form thereof according to claim 1.

29-36. (canceled)

37. A packaged pharmaceutical preparation comprising a pharmaceutical composition according to claim 26 in a container and instructions for using the composition to treat a patient suffering from anxiety, depression, a sleep disorder, attention deficit disorder, Alzheimer's dementia, or short-term memory loss.

38. (canceled)
